(19) United States
(12) Patent Application Publication
(10) Pub. No.: US 2009/0298708 A1
(43) Pub. Date: Dec. 3, 2009

(54) 2'-DEOXY-2'-FLUORO-BETA-D-ARABINONUCLEOSIDE 5'-TRIPHOSPHATES AND THEIR USE IN ENZYMATIC NUCLEIC ACID SYNTHESIS

(76) Inventors: Damha J. Masad, Quebec (CA); Peng Chang Geng, Montreal (CA)

Correspondence Address: OGILVY RENAULT LLP, 1, Place Ville Marie, SUITE 2500, MONTREAL, QC, H3B 1R1, CA

(21) Appl. No.: 12/438,286

(22) PCT Filed: Aug. 23, 2007
(86) PCT No.: PCT/CA2007/001515

§ 371 Date: Jun. 1, 2009

Related U.S. Application Data
(60) Provisional application No. 60/839,417, filed on Aug. 23, 2006.

(51) Int. Cl. C40B 30/04; C12P 19/34; C40B 50/06

(52) U.S. Cl. 506/9; 435/91.2; 506/26

(57) ABSTRACT

The invention relates to arabinose modified nucleoside 5′ triphosphates and to the biosynthesis and amplification of oligonucleotides containing and/or from templates containing arabinose modified nucleosides. The invention further relates to methods of generating modified oligonucleotide libraries for use in selection strategies, such as SELEX. The arabinose modified oligonucleotides of the invention are useful for modulating target nucleic acid expression, such as RNA.

### FIELD OF THE INVENTION

The invention relates to arabinose modified nucleoside 5′-triphosphates and the biosynthesis and amplification of oligonucleotides containing at least one arabinose modified nucleotide.

### BACKGROUND OF THE INVENTION

Oligonucleotide-based therapeutics have enormous potential for targeted therapy of cancer as well as inflammatory and infectious disease, exhibiting greater specificity and less toxicity than conventional chemotherapeutic drugs. The so-called “antisense” (AON) and “small interfering RNA” (siRNA) are the most prominent members of this class of agents. Both AONs and siRNAs are designed to bind to a specific sequence of an mRNA target through base-pairing interactions, thereby interfering with expression of the protein encoded by the mRNA. Scientists are hoping to use AONs and siRNAs to design new, more effective, drugs to inhibit gene expression and production of abnormal levels of cell proteins. [Stull, R. A. and Szoka, F. C. (1995) Pharmaceutical Research, 12, 465-483; Uhlmann E. and Peyman, A. (1990) Chemical Reviews, 90, 544-584.; Mittal, V. (2004) Nature Rev., 5, 355-365]. Nucleic acid “aptamers” are a recent addition to the large number of nucleic acid-derived molecules being investigated as potential therapeutics. AONs and siRNAs are designed to target a specific mRNA, whereas nucleic acid “aptamers” (from the Latin word “aptus”, meaning “to fit”) exert their effect by binding to a specific target protein thus blocking such protein from further function [Nimjee, S. M. et al. (2005) Annu. Rev. Med. 56, 555-83].

A powerful technique by which oligonucleotide aptamers are obtained is commonly referred to as SELEX, an acronym for Systematic Evolution of Ligands by Exponential Enrichment [Tuerk, C. and Gold, L. (1990) Science, 249, 505-510); Ellington, A. D. and Szostak, J. W. (1990) Nature, 346, 818-822]. This process begins by generating a very large “library” of randomized oligonucleotide sequences comprising 2′-deoxynucleotide (DNA) or ribonucleotide (RNA) units. Usually this library contains trillions of different oligonucleotide species that fold into unique three dimensional structures depending on the particular base sequence. The library is then incubated with the target protein of interest and, in a “selection” step, those oligonucleotides present in the library that bind the protein are separated from those that do not. The retained oligonucleotides are then amplified, typically by reverse transcriptase polymerase chain reaction (RT-PCR) and subsequently in vitro transcribed to generate a pool of oligonucleotides that have been enriched for those that bind the protein target of interest. Typically, these selection and amplification processes are repeated 8 to 15 times until the oligonucleotide aptamers with the highest affinity for the target protein are isolated. The “better fit” aptamers are then cloned and sequenced. Once identified, the aptamer is chemically synthesized on larger scale (typically via solid-phase synthesis) and its protein binding properties confirmed through in vitro assays.

Another interesting class of aptamers created by SELEX are the so-called “DNA enzymes” (DNAzyme) or “catalytic DNA”. These ligands bind to an RNA target instead of to a protein, and in the presence of a metal cofactor such as Mg⁺², catalyze the hydrolytic cleavage of such RNA into smaller fragments [Breaker, R. R. and Joyce, G. F. (1995) Chem. Biol., 2, 655-660; Santoro, S. W. and Joyce, G. F. (1997) Proc. Natl. Acad. Sci. USA, 94, 4262-4266]. Because DNA enzymes are capable of specifically modulating the biological function of their target RNA, they could have pharmaceutical utility and are recognized as potentially excellent drug leads or drug candidates [For a review see: Achenbach J. C. et al. (2004), Current Pharmaceutical Biotechnology, 5, 321-336].

Much progress towards clinical applications of aptamers has been made [Hicke, B. J. et al. (1996) J. Clin. Investig. 98, 2688-2692; Pietras, K. et al. (2002) Cancer Res. 62, 5476-5484; White, R. R. et al. (2003) Proc. Natl. Acad. Sci. U.S.A. 100, 5028-5033)]. Aptamers are receiving increasing attention, especially in view of the recent FDA approval of Macugen®, an RNA aptamer indicated for the treatment of neovascular age-related macular degeneration (AMD) [(a) Eyetech Study Group (2002)Retina, 22, 143-52; (b) Eyetech Study Group (2003) Opthalmology, 110, 979-86]. Nucleic acid aptamers have also been shown to control viral gene expression, including HIV gene expression, in vitro [(a) Sullenger B. A. et al. (1991) Journal of Virology, 65, 6811-6816; (b) Zimmermann K. et al. (1992) Human Gene Therapy, 3, 155-161; (c) Lee T. C. et al. (1992) New Biologist, 4, 66-74]. Aptamers may also prove useful for the treatment of other important human maladies, including infectious diseases, cancer, and cardiovascular disease.

Application of nucleic acid aptamers in vivo and their broader possible use as therapeutics, as with other oligonucleotide therapies, faces some key hurdles e.g., delivery, cellular uptake and biostability. There is a need to develop chemical modifications to produce clinically useful molecules. Initial work with antisense oligodeoxynucleotides (DNA) was carried out with unmodified, natural molecules. It soon became clear however, that native DNA was subject to relatively rapid degradation, primarily through the action of 3′ exonucleases, but as a result of endonuclease attack as well. Oligoribonucleotides (RNA) and siRNA are subject to the same considerations and are, in fact, generally more susceptible to nuclease degradation. Similar limitations could apply to nucleic acid aptamers where desirable. Given that the protein binding activity of aptamers is strongly dependent on the folding or 3D structure of the oligonucleotide strand, it is also highly desirable that such structure is of high thermal stability. The same is true for catalytic DNA aptamers (and analogs), which must bind to the RNA target with sufficient affinity to form a stable aptamer/RNA complex.

Until now, several methods have been devised to improve the stability of aptamers towards endo/exonucleases, most of which make use of SELEX and/or chemical synthesis. Nolte et al. have reported the identification of a mirror-design RNA aptamer (or “Spiegelmers”), which is acquired by selecting a normal RNA aptamer (D-RNA) against the enantiomer of a target protein, the mirror image of the target protein (D-amino acids), through the use of standard SELEX. When the resulting RNA aptamer (D-RNA) was converted to its enantiomeric form, L-RNA, with the same base composition, the L-RNA exhibited high binding affinity to the native protein molecule (L-amino acids) and high resistance against cleavage by nucleases. This strategy is limited to cases where an enantiomer of the target molecule is available [Nolte, A. et al. (1996) Nat. Biotechnol., 14, 1116-1119].

An alternative approach is the direct selection of an aptamer from libraries of modified oligonucleotides, typically modified RNAs (RNA SELEX procedure). Modifications must be chosen that are compatible with nucleic acid replicating enzymes such as reverse transcriptase or DNA and RNA polymerases [U.S. Pat. No. 5,660,985, entitled “High Affinity Nucleic Acid Ligands Containing Modified Nucleotides”, and U.S. Pat. No. 6,387,620, entitled “Transcription-free SELEX”]. The modifications most commonly used are those where the 2′—OH group of rU and rC is substituted with a 2′-fluoro or 2′-amino group. Such modification requires the availability of 2′-modified ribonucleoside 5′-triphosphates (2′-modified rNTPs) and their enzymatic incorporation by RNA polymerase. Following a round of selection, the aptamers are reverse transcribed into cDNA by a reverse transcriptase, and the cDNA obtained is amplified by PCR. 2′-Amino-modified nuclease-resistant aptamers have been selected to bind to autoantibodies of patients affected by the muscular disease myasthenia gravis. Such aptamers inhibit the binding of these autoantibodies to acetylcholine receptors on human cells, blocking the associated pathogenic consequences [Lee, S.-W. and Sullenger, B. A. (1997) Nat. Biotechnol., 15, 41-45]. Similarly, 2′-amino and 2′-fluoro-modified RNA aptamers directed against the human keratinocyte growth factor block its activity with high efficiency [Pagratis, N. C. et al. (1997) Nat. Biotechnol., 15, 68-73]. Further stabilization of RNA aptamers, if necessary, is done via post-SELEX chemical modifications, i.e., the winning RNA (or 2′-F/2′—NH₂ modified RNA) aptamer is synthesized chemically via gene machine synthesis introducing 2′-O-methylribonucleoside modifications at purine (rA and rG) sites that do not substantially change the binding properties of the aptamer. For example, Macugen®, an FDA approved aptamer for neovascular age-related macular degeneration (AMD), is a 2′F pyrimidine-containing RNA analogue in which all but two purine nucleosides are modified by 2′-OCH₃. The aptamer is further derivatized at the 5′-end with a polyethylene glycol segment to improve its pharmacokinetic properties, and at the 3′-end with a 3′p3′-dT cap to enhance resistance to 3′-exonucleases present in tissues or serum [(a) Eyetech Study Group (2002) Retina, 22, 143-52; (b) Eyetech Study Group (2003) Opthalmology, 110, 979-86].

It is noteworthy that most RNA aptamers evolved (via SELEX) for therapeutic use have either 2′-fluoro or 2′-amino groups within each pyrimidine ribonucleotide. This is because very few modified nucleoside triphosphates are compatible with the SELEX process, i.e., very few modified modified rNTPs are substrates of RNA polymerases and/or very few modified oligonucleotides are faithful templates for reverse transcriptase, both of which are key steps in the SELEX process. For this reason, the 2′-OMe ribonucleoside modification can only be incorporated post-SELEX via gene machine synthesis (the 2′-OMe rNTPs are not substrates of T7 RNA polymerase). To the best of our knowledge, in addition to 2′-fluoro and 2′-amino ribonucleoside 5′-triphosphates, very few other rNTPs are compatible with SELEX. Among the most promising analogues are:

(a) Ribonucleoside 5′-(alpha-P-borano)-triphosphates (BH₃-RNA) [Lato, S. M. (2002) Nucleic Acids Res., 30, 1401-1407].
(b) Ribonucleoside 5′-(alpha-thio)triphosphates (S-RNA) [Jhaveri, S. et al. (1998) Bioorg. Med. Chem. Lett., 8, 2285-2290].
(c) 2′-Deoxyribonucleoside 5′-(alpha-methyl)triphosphates (P-Me DNA) [Dineva, M. A. et al. (1993) Bioorg. Med. Chem., 1, 411-414].
(d) Alpha-L-Threofuranosyl nucleoside 5′-triphosphates (TNA) [Horhota, A. et al. (2005) J. Am. Chem. Soc., 127, 7427-7434].
(e) 4′-Thio-ribonucleoside 5′-triphosphates (4′S-RNA) [Kato, Y. et al. (2005) Nucleic Acids Res. 33, 2942-2951]

NTP derivatives (a), (b) and (c) have somewhat limited RNA polymerase-mediated RNA synthesis. Efficient oligonucleotide synthesis using TNA 5′-TP monomers (d) requires very specialized mutant DNA polymerases [Gardner, A. F. and Jack, W. E. (1999) Nucleic Acids Research, 27, 2545-2555; Gardner, A. F. and Jack, W. E. (2002) Nucleic Acids Research, 30, 605-613] that require carefully monitored conditions for achieving high-fidelity TNA synthesis [Ichida, J. K. (2005) Nucleic Acids Research, 33, 5219-5225]. Furthermore, DNA synthesis on a TNA template by more common polymerases such as Bst Pol I and MMLV reverse transcriptase required at least 1 hour to obtain full-length extension products. By comparison, TNA synthesis on a DNA template was even more challenging, requiring at least 1 day incubation with Deep Vent® (exo-) before the insertion of three contiguous TNA residues could occur [Chaput, J. C. et al. (2003) J. Am. Chem. Soc., 125, 856-857; Chaput, J. C. et al. (2003) J. Am. Chem. Soc., 125, 9274-9275]. Others have reported a similar finding for the incorporation of TNA 5′-TPs by Vent® (exo-) DNA polymerase, clearly showing that TNA 5′-TPs are not easily accepted by many common DNA polymerases [Kempeneers, V. et al. (2003) Nucleic Acids Res. 31, 6221-6226].

The general poor enzymatic recognition of modified nucleoside 5′-triphosphates underscores the difficulties involved in using DNA/RNA polymerases to synthesize unnatural polynucleotides. Accordingly, there is a need in the art to find alternative modified nucleoside 5′-triphosphates that are faithful substrates for DNA and RNA polymerases. Ideally, the resulting oligonucleotide incorporating such modified nucleotides should also serve as template for cDNA synthesis, so that the selection and amplification steps of the SELEX protocols could be applied.

2′-Deoxy-2′-fluoro-β-D-arabinonucleic acids (2′F-ANA or FANA) consist of 2′-Deoxy-2′-fluoro-β-D-arabinonucleotides (2′F-araN) having a modified arabinose sugar instead of the standard 2-deoxyribose or ribose sugars present in DNA and RNA, respectively. The structure also includes a fluorine atom at the 2′ carbon of the arabinose sugar, which confers upon the resulting FANA oligonucleotide unique characteristics compared to either DNA or RNA [Damha et al. (1998) J. Am. Chem. Soc., 121, 12976-12977]. For example, FANA is more resistant against degradative nucleases compared to the native DNA or RNA structure. In addition, FANA is capable of base-pairing with complementary DNA, RNA, and FANA oligonucleotides with high affinity [Wilds, C. J. and Damha, M. J. (2000) Nucleic Acids Res., 28, 3625-3635; Wilds, C. J., Ph.D. Thesis, McGill University, Montreal, 2000].

Although the synthesis of 2′-deoxy-2′-fluoro-5-methyl-arabinouridine (FMAU or 2′F-araT) 5′-triphosphate as well as its substrate properties for viral and human DNA polymerases have been reported previously [Ruth J. L and Cheng Y.-H. (1981) Mol. Pharm., 20, 415-422; Chiou, J.-F. and Cheng Y.-H. (1985) Antimicrobial Agents and Chemotherapy, 27, 416-418], there is no disclosure of an efficient, in vitro method for the biosynthesis of FANA and FANA-DNA strands, or the biosynthesis of DNA by FANA template-mediated polymerization.

In fact, the prior art suggests that fluoroarabinonucleoside 5′-triphosphate analogs are not suitable for in vitro selection/amplification processes. In the 1970-80's arabinonucleosides represented a class of potent antiviral and anticancer agents, each with a structural modification at one or two distinct sites (C5 at the base, or C2′ at the sugar). Pyrimidine arabinonucleoside analogues that seemed particularly promising for clinical use as antiherpetics included E-5-(2-bromovinyl)-1-β-D-arabinofuranosyluracil, 1-(2-deoxy-2-fluoro-β-D-arabinofuranosyl)-thymine (FMAU or 2′F-araT), 1-(2-deoxy-2-fluoro-β-arabinofuranosyl)-5-methylcytosine, and 1-(2-deoxy-2-fluoro-β-D-arabinofuranosyl)-5-iodocytosine. Two distinct possibilities were suggested for the cellular toxicity observed for the fluoroarabinonucleosides: once phosphorylated intracellularly to its active 5′-triphosphate form, they would have direct inhibitory effect on the viral or human DNA polymerases, and/or have an effect on DNA integrity/fidelity after incorporation into the growing DNA chain [Ruth J. L and Cheng Y.-H. (1981) Mol. Pharm., 20, 415-422; Sun, H. et al. (2005) Imaging DNA synthesis with [18F]FMAU and positron emission tomography in patients with cancer, Eur. J. Nucl. Med. Mol. Imag., 32, 15-22].

“Fialuridine” [1-(2-deoxy-2-fluoro-β-D-arabinofuranosyl)-5-iodouracil, or FIAU] was an antiviral agent with potent activity against hepatitis B virus (HBV) replication in vitro and in vivo. Biochemical studies that followed the failed clinical trial of this compound suggested that inhibition of mitochondrial DNA polymerase γ (DNA pol-γ) activity was largely responsible for the pathogenesis of the delayed mitochondrial toxicity observed [Lewis W. et al. (1994) Biochemistry, 33, 14620-14624]. FIAU 5′-TP is efficiently incorporated into DNA, but polymerase γ chain elongation is greatly impaired, possibly leading to increased inhibitory effects of this drug at sites of replication [Van Rompay, A. R. et al. (2003) Pharmacology and Therapeutics, 100, 119-139]. The inhibition constant (Kᵢ) with DNA pol-γ was 0.04 μM, the lowest Kᵢ (highest inhibition effect) among the other mammalian DNA polymerases studied [Lewis W. et al. (1994) Biochemistry, 33, 14620-14624]. Other mammalian DNA polymerases (pol α, β, ε, δ) can also insert 2′F-araTTP (FIAU 5′-TP) into duplex DNA, however, the oligonucleotide products formed in each case were significantly reduced in length compared to those generated in the presence of the native dTTP [Lewis W. et al. (1994) Biochemistry, 33, 14620-14624].

Clofarabine (2-fluoro-deoxy-9-β-D-arabinofuranosyl)-2-chloroadenine is a recently approved antileukemic nucleoside analog that was synthesized with the goal to combine the most favorable pharmacokinetic properties of fludarabine (2′F-araA) and cladribine (2Cl-dA) and at the same time to avoid the dose limiting neurotoxicity exhibited by some 2′-deoxy-2′-fluoroarabinonucleoside analogs. Minor chemical modifications in the structure of clofarabine (halogenation at the 2-position of adenine and substitution of a fluorine group at the C-2′position of the arabinose sugar) resulted in many of its characteristic features: (1) high affinity to deoxycytidine kinase (dCyd); (2) prolonged retention of clofarabine triphosphate in leukemic blasts; (3) inhibition of ribonucleotide reductase (RNR), an enzyme involved in maintaining intracellular nucleoside pools; and (4) potent inhibition of DNA synthesis and DNA chain termination [Parker, W. B. et al. (2003) Cancer Gene Ther., 10, 23-29; Carson, D. A. et al. (1992) Proc. Natl. Acad. Sci. USA., 89, 2970-2974; Xie C. et al. (1995) Cancer Res., 55, 2847-2852; Xie, K. C. and Plunkett, W. (1996) Cancer Res., 56, 3030-3037].

### SUMMARY OF THE INVENTION

Unexpectedly, it is shown that 2′F-araTTP (FMAUTP), 2′F-araCTP, 2′F-araATP and 2′F-araGTP, typically in the presence of native deoxynucleoside 5′-triphosphates (dNTPs), can be used in polymerization reactions to produce full-length DNA-FANA chimeric oligonucleotides in excellent yields. Also unexpectedly, it is shown that the biosynthesis of a DNA strand using a complementary chimeric DNA-FANA strand as template. Finally, it is shown that DNA-FANA strands can be synthesized on DANA-FANA templates, and the methodologies for realizing these capabilities are disclosed therein.

According to a broad aspect of the invention, there is provided a method for performing polymerase-directed oligonucleotide synthesis comprising: providing a template oligonucleotide; providing a primer for the template oligonucleotide; providing monomers of nucleoside-5′-triphosphates; and a polymerase, wherein at least one nucleoside of at least one of (i) the template oligonucleotide and (ii) the monomers is a 2′-deoxy-2′-fluoroarabinonucleoside (2′F-araN). Preferably the polymerase is one of Deep Vent® (“DV”), 9° Nm™ (“9N”) Bst, Taq, Phusion™ (“Ph”), Therminator™ (“Th”), Klenow Fragment (“Kf”), MMLV-RT (“MM” or “MMLV”) and HIV-1 RT (“HIV”).

In one preferable embodiment, the template oligonucleotide is DNA and at least one monomer is 2′F-araNTP.

In another preferable embodiment, the template oligonucleotide comprises at least one nucleoside that is 2′F-araN and the monomers are dNTPs.

According to another aspect of the invention, a method of performing SELEX is provided comprising providing a library of oligonucleotides, selecting the library for binding to a target molecule to produce a binding population and amplifying the binding populations. The amplifying step comprises a synthesis step that incorporates the above methods.

According to a still further aspect of the invention, there is provided a 2′F-araN (FANA) 5′-triphosphate of formula (I):

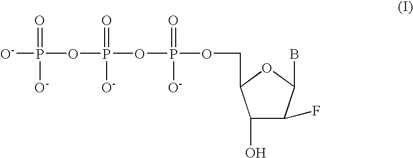

B is any base capable of Watson-Crick base pairing. Preferably, B is selected from the group consisting of thymine, uracil, cytosine, adenine, guanine, inosine, 2,6-diaminopurine, 5-methylcytosine, 5-fluorocytosine, 5-bromocytosine, 5-iodocytosine, isocytosine, N⁴-methylcytosine, 5-iodouracil, 5-fluorouracil, 4-thiouracil, 4-thiothymine, 2-thiouracil, 2-thiothymine, 7-deaza-adenine, N⁶-methyladenine, isoguanine, 7-deaza-guanine, and 6-thioguanine.

Advantageously, the above has significant implications in the investigation of FANA aptamers or FANA enzymes since efforts to evolve FANA aptamers or enzymes using SELEX would require enzymes capable of synthesizing and replicating (amplifying) FANA.

### BRIEF DESCRIPTIONS OF THE DRAWINGS

The invention will now be described in greater detail having regard to the appended drawings in which:

FIG. 1 illustrates various 2′F nucleotide triphosphates (NTPs). A. 1-(2-deoxy-2-fluoro-β-D-arabino-furanosyl)thymine 5′-triphosphate, sodium salt (2′F-araTTP); B. 1-(2-deoxy-2-fluoro-β-D-arabinofuranosyl)cytosine 5′-triphosphate, sodium salt (2′F-araCTP); C. 1-(2-deoxy-2-fluoro-β-D-ribo-furanosyl)uracil 5′-triphosphate, sodium salt (2′F-rUTP); D. 1-(2-deoxy-2-fluoro-β-D-ribofuranosyl) cytosine 5′-triphosphate, sodium salt (2′F-rCTP); E. 1-(2-deoxy-2-fluoro-β-D-arabinofuranosyl)adenosine 5′-triphosphate, sodium salt (2′F-araATP); F. 1-(2-deoxy-2-fluoro-β-D-arabinofuranosyl)guanosine 5′-triphosphate, sodium salt (2′F-araGTP).

FIG. 2 illustrates the polymerization of three contiguous 2′F-araT nucleotide units catalyzed by DNA polymerases on a DNA-template (PF22). The identity of the DNA polymerases, and the base sequence of the primer, DNA template (PF22) and resulting chimeric DNA-FANA oligonucleotide product are shown. A: All reactions at 37° C., 20 μL reaction volume, 1 μL enzyme added; B: reactions at 55° C., 2 μL Bst and 0.4 μL Ph enzymes in 20 μL reaction volume; H control was made by chain termination assay with 0.1 mM dATP, dGTP, dCTP, dTTP+ddTTP (1:1 molar ratio), 0.4 μL HIV in 20 μL reaction volume on the same template PF22

FIG. 3, illustrates the polymerization of three contiguous 2′F-araC nucleotide units catalyzed by DNA polymerases on a DNA-template (PF24). A: reactions at 37° C., 20 μL reaction volume, 1 μL enzyme, dNTPs and 2′F-araCTP: 0.33 mM for Kf and 0.5 mM for MMLV; B: reactions at 55° C., 40 μL reaction volume, 2 μL enzyme, 0.2 mM triphosphates; C: reactions at 55° C., 20 μL reaction volume, 2 μL Bst and 0.4 μL Ph enzymes, 0.2 mM triphosphates; products shown on lane H were formed through a chain termination assay with 0.1 mM dATP, dGTP, dTTP, dCTP+ddCTP (1:1 molar ratio), 0.4 μL HIV in 20 μL reaction volume, on the same template PF24.

FIGS. 4 A & B illustrate polymerization of six 2′F-araNTPs (3×2′F-araT and 3×2′F-araC) units catalyzed by DNA polymerases on a DNA-template PF32. The identity of the DNA polymerases, the primer, DNA template (PF32), and resulting chimeric DNA-FANA product are shown. A: 20 μL reaction volume for all, 1 μL enzyme in DV (group 1), 9N (group 3) and 0.4 μL enzyme in Ph (group 5); 2 μL enzyme in DV (group 2), 9N (group 4) and 0.4 μL enzyme in Ph (group 6), respectively; B: 40 μL reaction volume for HIV (group 1), Kf (group 3), Taq (group 5) and Bst (group 7) with 0.75 μL HIV, 2 μL Kf, 2 μL Taq and 2 μL Bst enzyme, respectively; 20 μL reaction volume and 2 μL corresponding enzyme for HIV (group 2), Kf (group 4), Taq (group 6) and Bst (group 8); triphosphate concentrations are shown above. Products shown on lane H were formed through a chain termination assay with 0.1 mM dATP, dGTP, dTTP+ddTTP (1:1 molar ratio), dCTP+ddCTP (1:1 molar ratio), 0.4 μL HIV in 20 μL reaction volume on the same template PF32.

FIGS. 5 A & B illustrate the polymerization of twelve fluoroarabinonucleotides (6×2′F-araT and 6×2′F-araC) units catalyzed by DNA polymerases on a DNA template (PF33). The identity of the DNA polymerases, the primer, DNA template (PF33), and resulting DNA-FANA product are shown. A: 20 μL reaction volume for all, 1 μL DV, 1 μL 9N, 0.4 μL Ph in group 1, 3, 5, respectively; 2 μL DV, 2 μL 9N, 0.4 μL Ph enzyme in group 2, 4, 6, respectively; B: 40 μL reaction volume with 0.75 μL HIV, 2 μL Kf, 2 μL Taq and 2 μL Bst enzyme in group 1, 3, 5, 7, respectively; 20 μL reaction volume with 1.5 μL HIV, 2 μL Kf, 2 μL Taq and 2 μL Bst enzyme in group 2, 4, 6, 8, respectively; NTP concentrations are shown above. Products shown on lane H1 were formed through a chain termination assay with 0.1 mM dATP, dGTP, dCTP, dTTP+ddTTP (1:1 molar ratio), 0.4 μL HIV in 20 μL reaction volume, on the same template PF33; products shown on lane H2 were formed through a chain termination assay with 0.1 mM dATP, dGTP, dTTP, dCTP+ddCTP (1:1 molar ratio), 0.4 μL HIV in 20 μL reaction volume on the same template PF33.

FIGS. 6 A & B illustrate the polymerization of fluoroarabinopyrimidine nucleotides (3×2′F-araT and 3×2′F-araC) units catalyzed by DNA polymerases on a chimeric (mixed) DNA-FANA template (40% FANA content after running start sequence, PF34), and resulting chimeric DNA-FANA product are shown. A: 20 μL reaction volume for all, 2 μL enzyme used; B: 20 μL reaction volume for all, 1.5 μL HIV in group 1, 2, 2 μL Kf, Taq, Bst in group 3-8, respectively; products shown on lane H1 were formed through a chain termination assay with 0.1 mM dATP, dGTP, dCTP, dTTP+ddTTP (1:1 molar ratio), 0.4 μL HIV in 20 μL reaction volume on the same template PF34; products shown on lane H2 were formed through a chain termination assay with 0.1 mM dATP, dGTP, dTTP, dCTP+ddCTP (1:1 molar ratio), 0.4 μL HIV in 20 μL reaction volume on the same template PF34.

FIGS. 7 A & B illustrate the polymerization of fluoroarabinopyrimidine nucleotides (6×2′F-araT and 6×2′F-araC) units catalyzed by DNA polymerases on a chimeric (mixed) DNA-FANA template (60% FANA content after running start sequence, PF35). The identity of the DNA polymerases, the primer, chimeric DNA-FANA template (PF35), and resulting chimeric DNA-FANA product are shown. A: 20 μL reaction volume for all, 2 μL DV, 9N and 0.5 μL Ph; B: 20 μL reaction volume for all, with 1.5 μL HIV, 2 μL Kf, 2 μL Taq and 2 μL Bst, respectively; products shown on lane H1 were formed through a chain termination assay with 0.1 mM DATP, dGTP, dCTP, dTTP+ddTTP (1:1 molar ratio), 0.4 μL HIV in 20 μL reaction volume on the same template PF35; products shown on lane H2 were formed through a chain termination assay with 0.1 mM dATP, dGTP, dTTP, dCTP+ddCTP (1:1 molar ratio), 0.4 μL HIV in 20 μL reaction volume on the same template PF35.

FIGS. 8 A & B illustrate the activity of FANA as a template for polymerase-directed DNA synthesis (FANA template PF31). The identity of the DNA polymerases, the base sequence of the primer, chimeric DNA-FANA template (PF31), and resulting DNA oligonucleotide product are shown. Reaction volume was 30 μL, and dNTP and enzyme amounts/concentrations are also shown. Bottom figure (B): an magnified gel picture of 8A (top) to show bands resulting from the observed dNTP incorporations in more detail; P is a full-length product control.

FIGS. 9 A & B illustrate the incorporation of multi 2′F-araA or 2′F-araG nucleotide units (A: 8×2′F-araA; B: 11× 2′F-araG) catalyzed by DNA polymerases on a DNA-FANA chimeric template (PF34). The identity of the DNA polymerases, and the base sequence of the primer, FANA-DNA template (PF34) and resulting chimeric DNA-FANA oligonucleotide product are shown. A: Incorporation of 8×2′F-araA; reactions at 55° C. (except for Kf at 37° C.), 20 μL reaction volume, 2 μL enzyme added (except for 1 uL Bst and Taq); 0.4 mM 2′F-araATP plus dTTP, dCTP and dGTP; P: full-length product control, made from 0.2 mM dNTPs and 1 μL DV in 20 μL reaction volume on a DNA template PF32 with same sequence of PF34; H control was formed through a chain termination assay with 0.1 mM dATP, dGTP, dCTP, dTTP+ddTTP (1:1 molar ratio), 0.3 μL HIV in 20 μL reaction volume, on a DNA template PF32 with same sequence of PF34. B: Incorporation of 11× 2′F-araG; reactions at 55° C. (except for Kf at 37° C.), 20 μL reaction volume, 2 μL enzyme added (except for 1 uL Bst and Taq); 0.4 mM 2′F-araGTP plus dTTP, dCTP and DATP; P: full-length product control, made from 0.2 mM dNTPs and 1 μL DV in 20 μL reaction volume on a DNA template PF32 with same sequence of PF34; H control was formed through a chain termination assay with 0.1 mM DATP, dGTP, dCTP, dTTP+ddTTP (1:1 molar ratio), 0.3 μL HIV in 20 μL reaction volume, on a DNA template PF32 with same sequence of PF34.

FIG. 10 illustrates the incorporation of 2′F-araN units (7×2′F-araG, 11× 2′F-araA, 6×2′F-araT, 6×2′F-araC) catalyzed by DNA polymerases on a DNA template (PF33). Primer extension reaction conditions were performed with 100 nM primer/template (1:1 molar ratio), 0.2 mM 2′F-araNTPs (shown above) and 1 μL DV, 9N, Th, Bst and Taq, KF, 0.5 μL Ph and MMLV, respectively in 20 μL reaction volume, at 55° C. (DV, 9N, Th, Bst, Taq, Ph) or 37° C. (KF and MMLV). Reaction progress over time was analyzed by 12% denaturing PAGE. Lane P is the product control obtained in a similar primer extension assay with 1 μL DV, 0.2 mM dNTPs at 55° C. for 30 min, on the same template PF33; PF39 is a 27-nt DNA control.

FIG. 11A illustrates the fidelity of 2′F-araATP incorporation on a DNA template PF41 by DV and 9N DNA polymerases. Reaction conditions: 0.8 unit DV and 9N, 25 μM triphosphates at 55° C. in 30 μL reaction volume; incubation time: 30 min. Group 1 & 5: dNTPs; 2 & 6: dropout of dATP; 3 & 7: 2′F-araNTPs, 4 & 8: dropout of 2′F-araATP; 27mer control DNA is PF39.

FIG. 11B illustrates the fidelity of 2′F-araGTP incorporation on DNA template PF43 by DV and 9N DNA polymerases. Reaction conditions: primer/template (1:1 molar ratio): 100 nM; 0.4 μL (0.8 unit) DV, 9N, 25 μM triphosphate concentration at 55° C. in 30 μL reaction volume. Reaction time for dNTPs (group 1, 5) was 30 min and others were shown above. Group 1 & 5: dNTPs; 2 & 6: drop out dGTP (i.e. dTTP, dCTP and dATP), 3 & 7: 2TF-araNTPs, 4 & 8: drop out 2′F-araGTP; lane P is the template PF41 as the full-length product control; PF39 is a 27-nt DNA control. All samples were analyzed by 12% denaturing PAGE.

FIG. 12A illustrates the fidelity of 2′F-araTTP incorporation by DV and 9N DNA polymerases on a DNA template PF21. Reaction conditions: 100 nM primer/template (1:1 molar ratio); 0.4 μL (0.8 unit) DV or 9N, 25 μM triphosphate concentration at 55° C. in 30 μL reaction volume. Reaction time for dNTPs (group 1, 5) was 30 min and others were shown above. Groups 1 & 5: dNTPs; 2 & 6: drop out dTTP; 3 & 7: 2′F-araNTPs; 4 & 8: drop out 2′F-araTTPs. in B: group 1 & 5: dNTPs; 2 & 6: drop out dCTP; 3 & 7: 2′F-araNTPs; 4 & 8: drop out 2′F-araCTPs; PF39 is a 27-nt DNA control. All samples were analyzed by 12% denaturing PAGE.

FIG. 12B illustrates the fidelity study of 2′F-araCTP incorporation by DV and 9N DNA polymerases on a DNA template PF23. Reaction conditions: 100 nM primer/template (1:1 molar ratio); 0.4 μL (0.8 unit) DV or 9N, 25 μM triphosphate concentration at 55° C. in 30 μL reaction volume. Reaction time for dNTPs (groups 1, 5) was 30 min and others were shown above. Groups 1 & 5: dNTPs; 2 & 6: drop out dCTP; 3 & 7: 2′F-araNTPs; 4 & 8: drop out 2′F-araCTPs; PF39 is a 27-nt DNA control. All samples were analyzed by 12% denaturing PAGE.

FIG. 13 illustrates the fidelity of 2′F-araNTP incorporation on pyrimidine rich DNA template PF21 by Ph DNA polymerase. Reaction conditions: 0.8 unit Ph, 25 μM triphosphate at 55° C. in 30 μL reaction volume for 30 min. lane 1: dNTPs; 2: drop out dCTP (i.e. dATP, dGTP and dTTP, shorten as AGT; same coding for lane 3-5), lane 6: 2′F-araNTPs; 7: drop out 2′F-araCTP (i.e. 2′F-araATP, 2′F-araGTP, 2′F-araTTP, shorten as AGT; same coding for lane 8-10). Template PF41 and primer PF20 are shown; PF39 is a 27-nt DNA control.

FIG. 14 illustrates the fidelity of 2′F-araNTP incorporation on purine rich DNA template PF41 by Ph DNA polymerase. The base sequence of the primer, DNA template and resulting terminated products in different dropout conditions (condition 1-5) is shown. Primer extension conditions: 100 nM primer/template (1:1 molar ratio); for dNTPs (lanes 1-5): 0.4 μL (0.8 unit) Ph, 25 μM dNTP concentration; for 2′F-araNTPs (lanes 6-10): 0.8 μL (1.6 unit) Ph, 0.2 mM 2′F-araNTP at 55° C. in 30 μL reaction volume for 30 min. Lane 1: dNTPs; 2: drop out dCTP (i.e. dATP, dGTP and dTTP, shorten as AGT, same coding for lanes 3-5), lane 6: 2′F-araNTPs; 7: drop out 2′F-araCTP (i.e. 2′F-araATP, 2′F-araGTP, 2′F-araTTP, shorten as AGT; same coding for lanes 8-10). The template PF41 and the primer PF20 are shown; PF39 is a 27mer DNA control. All samples were analyzed by 12% denaturing PAGE.

FIG. 15 illustrates the efficiency of incorporation of 2′F-araT versus 2′F-rU on DNA template PF22 by MMLV-RT DNA polymerase (20 units). A: Reactions conducted at 37° C., in a 60 μL reaction volume, with 1 μL (20 units) enzyme; triphosphate concentrations were shown; products shown on lane H were formed through a chain termination assay with 0.1 mM dATP, dGTP, dCTP, dTTP+ddTTP (1:1 molar ratio) on the template PF22, 0.4 μL HIV in 20 μL reaction volume at 37° C. B: A plot of time-course of full-length product (%). P: full-length product control.

FIG. 16 illustrates the efficiency of incorporation of 2′F-araT versus 2′F-rU on DNA template PF22 by MMLV-RT DNA polymerase (12 units). The base sequence of the primer, DNA template (PF22) and resulting chimeric DNA-2F′-ANA oligonucleotide product are shown. A: Reactions conducted at 37° C., in a 40 μL reaction volume, with 0.6 μL (12 units) enzyme; triphosphate concentrations shown; products shown on lane H were formed through a chain termination assay with 0.1 mM dATP, dGTP, dCTP, dTTP+ddTTP (1:1 molar ratio) on the same template PF22, at 37° C. B: A plot of time-course of full length products with the second incorporations of 2′F-araTTP, 2′F-rUTP or rUTP (%).

FIG. 17 illustrates A: the efficiency of incorporation of 2′F-araT versus 2′F-rU on DNA template PF22 by 9N DNA polymerase; and B: the efficiency of incorporation of 2′F-araC versus 2′F-rC on DNA template PF24 by 9N DNA polymerase; and C: a plot of time-course of full-length product (%). Reactions conducted at 55° C. in a 60 μL reaction volume, with 3 μL (6 units) 9N DNA polymerase; triphosphate concentrations are shown; products shown in lane H were formed through a chain termination assay with 0.1 mM dATP, dGTP, dCTP, dTTP+ddTTP (1:1 molar ratio), 0.4 μL HIV in 20 μL reaction volume on the template PF22 at 37° C.; products shown in lane M were formed through a chain termination assay with 0.2 mM dATP, dGTP, dTTP, dCTP+ddCTP (1:1 molar ratio), 0.4 μL (8 units) MMLV-RT in 20 μL reaction volume on the template PF24 at 37° C. C: A plot of time-course of full-length product (%). P: full-length product control.

FIG. 18 illustrates the efficiency of incorporation of 2′F-araT versus 2′F-rU through DNA template (PF22)-mediated HIV DNA polymerization. A: Reactions conducted at 37° C. in a 40 μL reaction volume, with 0.66 μL (18 units) enzyme; products shown on lane H were formed through a chain termination assay with 0.1 mM dATP, dGTP, dCTP, dTTP+ddTTP (1:1 molar ratio), 0.4 μL HIV in 20 μL reaction volume on the same template PF22 at 37° C. B: A plot of time-course of full-length product (%).

FIG. 19 illustrates the efficiency of incorporation of 2′F-araC versus 2′F-rC through DNA template PF24 mediated by MMLV-RT DNA polymerization. A: Reactions conducted at 37° C. in a 40 μL reaction volume, with 0.6 μL (12 units) MMLV-RT DNA polymerase; triphosphate concentrations are shown; products shown in lane H were formed through a chain termination assay with 0.1 mM dATP, dGTP, dTTP, dCTP+ddCTP (1:1 molar ratio) on the PF24 template, 0.4 μL MMLV-RT RT in 20 μL reaction volume at 37° C. B: A plot of time-course of full-length product (%). P: full-length product control.

FIG. 20 illustrates the efficiency of incorporation of 2′F-araC versus 2′F-rC through DNA template (PF24)-mediated HIV polymerization assay. A: Reactions at 37° C., 40 μL reaction volume, 0.66 μL (18 units) enzyme; products shown on lane H were formed through a chain termination assay with 0.1 mM dATP, dGTP, dTTP, dCTP+ddCTP (1:1 molar ratio), 0.4 μL HIV in 20 μL reaction volume on the same template PF22 at 37° C. B: A plot of time-course of full-length product (%).

FIG. 21 illustrates A: the efficiency of incorporation of 2′F-T versus 2′F-rU on DNA template PF22 by DV DNA polymerase; B: the efficiency of incorporation of 2′F-araC compared to 2′F-rC on DNA template PF24 by DV DNA polymerase; and C: a plot of time-course of full-length product. Reactions conducted at 55° C. in a 60 μL reaction volume, with 3 μL (6 units) DV DNA polymerase; triphosphate concentrations are shown; products shown in lane H were formed through a chain termination assay with 0.1 mM dATP, dGTP, dCTP, dTTP+ddTTP (1:1 molar ratio), 0.4 μL HIV in 20 μL reaction volume on the template PF22 at 37° C.; products shown in lane M were formed through a chain termination assay with 0.2 mM dATP, dGTP, dTTP, dCTP+ddCTP (1:1 molar ratio), 0.4 μL MMLV-RT in 20 μL reaction volume on the template PF24 at 37° C. P: full-length product control.

FIG. 22 illustrates A: the efficiency of incorporation of dATP and dGTP in the presence of various pyrimidine NTP combinations, namely: 2′F-araTTP/2′F-araCTP (group 3); 2′F-araTTP/2′F-rCTP (group 4); 2′F-rUTP/2′F-araCTP (group 5); 2′F-rUTP/2′F-rCTP (group 6); and rUTP/rCTP (group 7). Reactions were carried out at 55° C.; 40 μL reaction volume, 9N enzyme (2 μL; 4 units); 0.2 mM TPs; products shown on lane H were formed through a chain termination assay with 0.1 mM dATP, dGTP, dCTP, dTTP+ddTTP (1:1 molar ratio), 0.4 μL HIV in 20 μL reaction volume on the same template PF32. Bottom figure B: A plot of time-course of full-length product (%).

FIG. 23 illustrates A: the efficiency of incorporation of dATP, dGTP on DNA template PF32 by DV DNA polymerase in the presence of various pyrimidine NTP combinations, namely: 2′F-araTTP/2′F-araCTP (group 3); 2′F-araTTP/2′F-rCTP (group 4); 2′F-rUTP/2′F-araCTP (group 5); 2′F-rUTP/2′F-rCTP (group 6); and rUTP/rCTP (group 7). Reactions were carried out at 55° C.; 40 μL reaction volume, DV enzyme (2 μL; 4 units); 0.2 mM triphosphates; products shown on lane H were formed through a chain termination assay with 0.1 mM dATP, dGTP, dCTP, dTTP+ddTTP (1:1 molar ratio), 0.4 μL HIV in 20 μL reaction volume on the same template PF32. B: A plot of time-course of full-length product (%).

FIG. 24 illustrates A: the efficiency of incorporation of dATP, dGTP on DNA template PF32 by HIV DNA polymerase in the presence of various pyrimidine NTP combinations, namely: 2′F-araTTP/2′F-araCTP (group 3); 2′F-araTTP/2′F-rCTP (group 4); 2′F-rUTP/2′F-araCTP (group 5); 2′F-rUTP/2′F-rCTP (group 6); and rUTP/rCTP (group 7). Reactions were carried out at 37° C.; 40 μL reaction volume, HIV enzyme (1 μL; 27.3 units, except 0.4 μL for the first group); 0.2 mM triphosphates (except for 0.1 mM dNTPs in the first group); products shown on lane H were formed through a chain termination assay with 0.1 mM dATP, dGTP, dCTP, dTTP+ddTTP (1:1 molar ratio), dCTP+ddCTP (1:1 molar ratio), HIV in 20 μL reaction volume on the same template PF32 at 37° C. for 15 minutes; P: full-length product control. B: A plot of time-course of full-length product (%).

### DETAILED DESCRIPTION

In the following description, numerous specific details are set forth to provide a thorough understanding of the invention. However, a person skilled in the art will understand, given the context, that circumstances exist in which the invention may be practiced without specific preferred features. In the following description reference is made to certain terms of the art.

“Nucleosides” are individual units consisting of a heterocyclic base covalently bonded to a 5-carbon sugar. The base is any heterocyclic base capable of Watson-Crick base pairing and includes any one of the natively found purine and pyrimidine bases, adenine (A), thymine (T), cytosine (C), guanine (G) and uracil (U), but also any modified or analogous forms thereof. Examples of non-naturally occurring bases that are capable of forming base-pairing relationships include, but are not limited to, aza and deaza pyrimidine analogues, aza and deaza purine analogues, and other heterocyclic base analogues, wherein one or more of the ring atoms and functional groups of the purine and pyrimidine rings have been substituted by heteroatoms, e.g., carbon, fluorine, oxygen, sulfur, and the like. Preferably, bases include, but are not limited to, inosine, 2,6-diaminopurine, 5-methylcytosine, 5-fluorocytosine, 5-bromocytosine, 5-iodocytosine, isocytosine, N⁴-methylcytosine, 5-iodouracil, 5-fluorouracil, 4-thiouracil, 4-thiothymine, 2-thiouracil, 2-thiothymine, 7-deaza-adenine, N⁶-methyladenine, isoguanine, 7-deaza-guanine, and 6-thioguanine. The 5-carbon sugar will typically be a naturally occurring sugar such as 2-deoxyribose, ribose or arabinose, but can be any 5-carbon sugar or modified form thereof, including but not limited to 2-deoxy-2-fluoro-ribose, 2-deoxy-2-fluoroarabinose or even carbocyclic sugars where a carbon function is substituted for the oxygen atom in the sugar ring (i.e., 6 carbon analog).

“Nucleotides” are nucleoside units further having a phosphorus moiety covalently bonded to the sugar moiety of the nucleoside at either the 3′ or the 5′ position of the sugar.

Bases are also denoted “N” in reference to the abbreviation of nucleoside or nucleotide, including but not limited to instances such as “dNTP” or “2′F-araNTP”.

“Modified nucleotide” refers to a nucleotide that differs from a naturally occurring nucleotide in some modification and can be made by chemical modification of the phosphate backbone, sugar unit or nucleoside base. Preferable modified nucleotides include, but are not limited to ribonucleoside 5′-(alpha-P-borano)-triphosphates (BH₃-RNA), ribonucleoside 5′-(alpha-thio)triphosphates (S-RNA), 2′-deoxyribonucleoside 5′-(alpha-methyl)triphosphates (P-Me DNA), alpha-L-threofuranosyl nucleoside 5′-triphosphates (TNA), 4′-thio-ribonucleoside 5′-triphosphates (4′S-RNA), 2′-amino-ribonucleoside-5′triphosphate (2′NH₂-RNA), and 2′-deoxy-2′-fluororibonucleoside-5′-triphosphate (2′F-RNA).

Where at least two modified nucleotides are linked, a modified oligonucleotide results. Representative modifications include phosphorothioate, phosphorodithioate, methyl, phosphonate, phosphotriester or phosphoramidate inter-nucleoside linkages in place of phosphodiester inter-nucleoside linkages; deaza or aza purines and pyrimidines in place of natural purine and pyrimidine bases, pyrimidine bases having substituent groups at the 5 or 6 position; purine bases having altered substituent groups at the 2, 6 or 8 positions or 7 position as 7-deazapurines; sugars having substituent groups at, for example, their 2′ position; or carbocyclic or acyclic sugar analogs and additionally include arabinonucleotides or modified arabinonucleotide residues, in particular 2′-fluoro-substituted arabinonucleotides. Such modified oligonucleotides are best described as being functionally interchangeable with, yet structurally different from, natural oligonucleotides. In addition, modification can be made wherein nucleoside units are joined through groups that substitute for the internucleoside phosphate or sugar phosphate linkages.

“Oligonucleotides”, or “oligomers”, are polymers of at least two nucleoside units, wherein each of the individual nucleoside units is covalently linked to at least one other nucleoside unit through a single phosphorus moiety. In the case of naturally occurring oligonucleotides, the covalent linkage between nucleoside units is a phosphodiester bond. Nevertheless, the term “oligonucleotide” as used herein includes, but are not limited to, oligonucleotides that are modified with respect to any one or more of the following: (1) the phosphodiester bond between nucleoside units, (2) the individual nucleoside units themselves and/or (3) the ribose, or sugar, moiety of the nucleoside units. Further, “oligonucleotides” include ribonucleic acid (RNA) sequences, deoxyribonucleic acid (DNA) sequences and arabinonucleic acid (ANA) of more than one nucleotide in either single chain or duplex form. The term includes polymers of ribonucleotides, arabinonucleotides and deoxyribonucleotides with the ribonucleotide, arabinonucleotides and/or deoxyribonucleotides being connected together via 5′ to 3′ linkages. However, linkages may include any of the linkages known in the nucleic acid synthesis art including, for example, nucleic acids comprising 5′ to 2′ linkages. Oligonucleotides as defined herein are comprised of about 1 to about 100 nucleotides, more preferably from 1 to 80 nucleotides, and even more preferably from about 15 to about 60 nucleotides.

2′F-araN (FANA) 5′-triphosphates of the general formula (I):

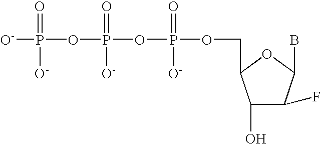

were generated wherein B is adenosine, cytosine, guanine and thymine. The resulting 2′-ara-adenosine 5′-triphosphates, 2′-ara-cytosine 5′-triphosphates, 2′-ara-guanine 5′-triphosphates and 2′-ara-thymine 5′-triphosphates were incorporated into template oligonucleotides or used in the oligonucleotide synthesis reactions as described below. It will be understood by a person skilled in the art that B may additionally be any base capable of Watson-Crick base-pairing. As such, B could additionally include, without limitation, uracil or a modified base including, but not limited to, inosine, 2,6-diaminopurine, 5-methylcytosine, 5-fluorocytosine, 5-bromocytosine, 5-iodocytosine, isocytosine, N 4-methylcytosine, 5-iodouracil, 5-fluorouracil, 4-thiouracil, 4-thiothymine, 2-thiouracil, 2-thiothymine, 7-deaza-adenine, N⁶-methyladenine, isoguanine, 7-deaza-guanine, and 6-thioguanine.

Unexpectedly, it is shown that 2′F-araTTP (FMAUTP), 2′F-araCTP, 2′F-araATP and 2′F-araGTP, typically in the presence of native deoxynucleoside 5′-triphosphates (dNTPs), can be used in polymerization reactions to produce full-length DNA-FANA chimeric oligonucleotides in excellent yields. Also unexpectedly, it is shown that the biosynthesis of a DNA strand using a complementary chimeric DNA-FANA strand as template. Finally, it is shown that DNA-FANA strands can be synthesized on DANA-FANA templates, and the methodologies for realizing these capabilities are disclosed therein.

According to a broad aspect of the invention, there is provided a method for performing polymerase-directed oligonucleotide synthesis comprising: providing a template oligonucleotide; providing a primer for the template oligonucleotide; providing monomers of nucleoside-5′-triphosphates; and a polymerase, wherein at least one nucleoside of at least one of (i) the template oligonucleotide and (ii) the monomers is a 2′-deoxy-2′-fluoroarabinonucleoside (2′F-araN). Preferably the polymerase is one of Deep Vent® (“DV”), 9°Nm™ (“9N”) Bst, Taq, Phusion™ (“Ph”), Therminator™ (“Th”), Klenow Fragment (“Kf”), MMLV-RT (“MM” or “MMLV”) and HIV-1 RT (“HIV”).

In some embodiments, the template oligonucleotide is DNA.

In some embodiments, the at least one monomer is 2′F-araNTP.

In some embodiments, the at least one monomer is dNTP.

In some embodiments, all monomers, or ones having a predetermined base, are 2′F-araNTPs.

In some embodiments, the monomers are a mixture of dNTPs and 2′F-araNTPs.

In some embodiments, the template oligonucleotide comprises at least one nucleoside that is 2′F-araN. In other embodiments, the template oligonucleotide consists of FANA.

In some embodiments, the template oligonucleotide is a FANA-DNA chimera with a FANA percentage less than 100%, 60% or 40%.

In some embodiments, the monomers comprise at least one at least one modified nucleoside 5′-triphosphate. Preferably, the at least one modified nucleoside triphosphate is selected from the group consisting of ribonucleoside 51-(alpha-P-borano)-triphosphates (BH₃-RNA), ribonucleoside 5′-(alpha-thio)triphosphates (S-RNA), 2′-deoxyribonucleoside 5′-(alpha-methyl)triphosphates (P-Me DNA), alpha-L-threofuranosyl nucleoside 5′-triphosphates (TNA), 4′-thio-ribonucleoside 5′-triphosphates (4′S-RNA), 2′-amino-ribonucleoside-5′triphosphate (2′NH₂-RNA), 2′-deoxy-2′-fluororibonucleoside-5′-triphosphate (2′F-RNA) and combinations thereof.

In some embodiments, the 2′F-araN unit comprises any heterocyclic base capable of Watson-Crick base pairing. Preferably, the base is selected from the group consisting of thymine, uracil, cytosine, adenine, guanine, inosine, 2,6-diaminopurine, 5-methylcytosine, 5-fluorocytosine, 5-bromocytosine, 5-iodocytosine, isocytosine, N⁴-methylcytosine, 5-iodouracil, 5-fluorouracil, 4-thiouracil, 4-thiothymine, 2-thiouracil, 2-thiothymine, 7-deaza-adenine, N⁶-methyladenine, isoguanine, 7-deaza-guanine, 6-thioguanine, and combinations thereof. More preferably, B is selected from the group consisting of thymine, uracil, cytosine, adenine, and guanine.

According to another aspect, a library of oligonucleotides is generated using the methods of the invention.

According to another aspect of the invention, a method of performing SELEX is provided comprising providing a library of oligonucleotides, selecting the library for binding to a target molecule to produce a binding population and amplifying the binding populations. The amplifying step comprises a synthesis step that incorporates the above methods. In some embodiments, the library of oligonucleotides is a library of the present invention.

According to a still further aspect of the invention, there is provided a 2′F-araN (FANA) 5′-triphosphate of formula (I):

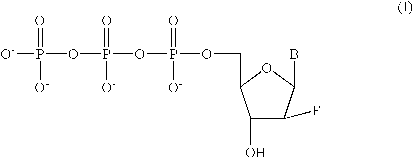

B is any base capable of Watson-Crick base pairing. Preferably, B is selected from the group consisting of thymine, uracil, cytosine, adenine, guanine, inosine, 2,6-diaminopurine, 5-methylcytosine, 5-fluorocytosine, 5-bromocytosine, 5-iodocytosine, isocytosine, N⁴-methylcytosine, 5-iodouracil, 5-fluorouracil, 4-thiouracil, 4-thiothymine, 2-thiouracil, 2-thiothymine, 7-deaza-adenine, N⁶-methyladenine, isoguanine, 7-deaza-guanine, and 6-thioguanine. More preferably, B is selected from the group consisting of thymine, uracil, cytosine, adenine, and guanine.

Primer extension assays were performed. DNA and FANA-DNA templates and primers used in this study are shown in Table 1. The oligomers were modeled after those reported by Chaput et al. and Kato et al. [Chaput, J. C. et al. (2003) J. Am. Chem. Soc., 125, 856-857; Chaput, J. C. et al. (2003) J. Am. Chem. Soc., 125, 9274-9275; Kato, Y. et al. (2005) Nucleic Acids Res. 33, 2942-2951]. The oligomers were designed to test the incorporation of three 2′F-araT units (PF22), three 2′F-araC units (PF24), six mixed 2′F-araT and 2′F-araC units (PF32), 12 mixed 2′F- and 2′F-araC units (PF33), and multiple 2′F-araA and 2′F-araG units; they also include DNA-FANA chimeras, i.e., PF34 (40% 2′F-araN content), PF35 (60% 2′F-araN content) and PF31 (100% 2′F-araN content).

Various different types of DNA polymerases were assessed, including the mesophilic enzyme Klenow fragment of DNA pol I (3′→5′ exo-) (“Kf”); the thermophilic DNA polymerases DV, 9N, Taq, Bst pol I, Th and Ph; and the viral reverse transcriptases HIV RT and MMLV. Kf is a genetically engineered proteolytic product of E. coli pol A gene that encodes DNA polymerase I which retains polymerase activity but has a mutation which abolishes the 3′->5′ and 5′->3′ exonuclease activities. DV is a genetically engineered DNA polymerase purified from a strain of E. coli that carries the DV DNA polymerase gene from Pyrococcus species GB-D. The gene has been engineered to eliminate 3′->5′ proofreading exonuclease activity. 9N is a genetically engineered DNA polymerase that has decreased 3′->5′ proofreading exonuclease activity. 9N is purified from a strain of E. coli that carries a modified 9° N DNA polymerase gene from Thermococcus species (strain 9° N-7). Taq DNA polymerase is a thermostable DNA polymerase that possesses a 5′->3′ polymerase activity and a double-strand specific 5′->3′ exonuclease activity. Taq polymerase is isolated from E. coli engineered to express Taq polymerase from Thermus aquaticus YT-1. Bst DNA polymerase (Large fragment) is a genetically engineered polymerase that consists of the portion of the Bacillus stearothemmophilus DNA polymerase protein responsible for 5′->3′ polymerase activity but lacks the 5′->3′ exonuclease domain. Th DNA polymerase is a genetically engineered, archaeon-derived DNA polymerase that is a variant of the 9N polymerase described above. The recombinant protein is isolated from E. coli engineered to express 9° NA485L (exo-) DNA polymerase gene, a genetic variant of the native DNA polymerase from Thermococcus species 9° N-7. Ph is a recombinant DNA polymerase that consists of a novel Pyrococcus-like enzyme fused with a double-strand DNA-binding and processivity-enhancing domain.

### Template-directed Oligomerization of 2′F-araNTPs

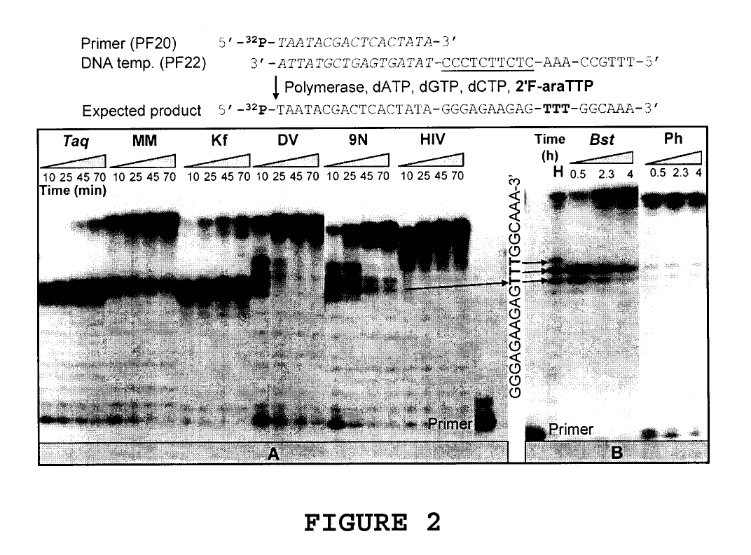

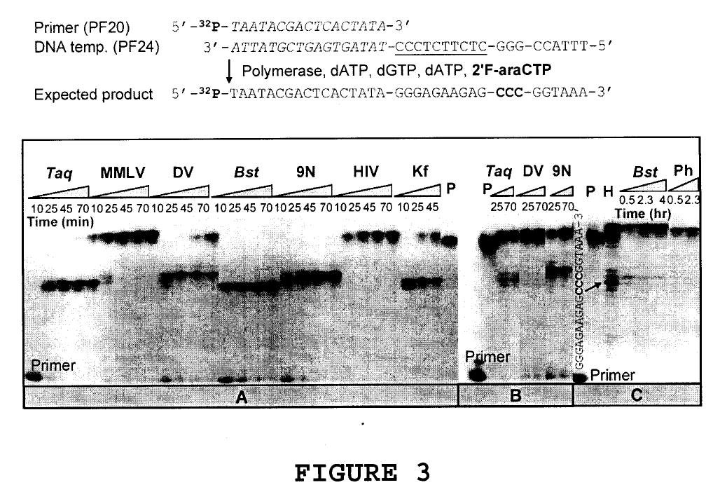

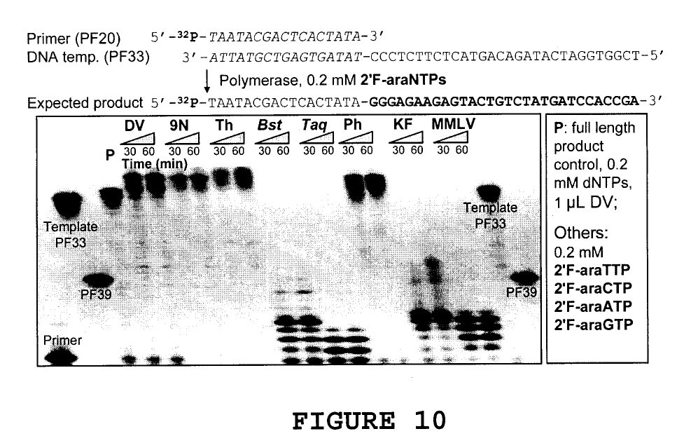

Primer extension assays were used to assess the incorporation of 2′F-araNTP on the various DNA templates (Table 1). The DNA primer (PF20) was first radioactively labeled at the 5′-hydroxyl terminus with a radioactive phosphorus probe and the enzyme T4 polynucleotide kinase (T4 PNK) (see Example 1 for General Methods). To assess the incorporation of 2′F-araNTPs, the primer and template were annealed, and the nucleoside 5′-triphosphates added in the presence of enzyme under the conditions specified in Table 2. Time points were obtained by analyzing small aliquots from the reaction mixture through polyacryamide gel electrophoresis (PAGE) and autoradiography. Results from template-directed oligomerization on DNA template PF22 are given in FIGS. 2, 3 and 10. Full-length product is determined by comparison to the results from all natural dNTPs as seen in lane H of FIGS. 2 and 3 and lane P of FIG. 10.

The data from FIG. 2 indicates successful incorporation of 2′F-araTTP opposite to a template dA residue and also show that: (a) DNA polymerases are able to incorporate three contiguous 2′F-araT nucleotide units on DNA-template PF22; (b) incorporation of 2′F-araTTP proceeds well, despite some pausing observed after addition of the first 2′F-araT unit. The precise site of “pausing” is determined by conducting another assay in which 2′F-araTTP is replaced with the chain terminator ddTTP in the presence of dTTP (lane H; FIG. 2). Remarkably, the thermophilic enzymes DV, 9N and Taq were able to incorporate three 2′F-araTTP units, even at non-optimum temperatures (37° C.), providing the full length product. Polymerase Kf was able to incorporate 2′F-araTTP even at lower concentrations (0.033 mM) (FIG. 2).

Slightly different results were obtained with 2′F-araCTP (FIG. 3). The thermophilic enzymes DV, 9N, Taq and Bst could incorporate one 2′F-araCTP and only DV and Taq could incorporate three 2′F-araCTP at 37° C. (data not shown). At a higher temperature (55° C.), 9N and Bst (and even Ph) were able to incorporate three 2′F-araC units and gave the expected full-length products (FIG. 3). The mesophilic and retroviral enzymes gave full-length products, with some pausing detected after incorporation of the first 2′F-araC unit (37° C.).

Primer extension assays further showed incorporation of each of 2′F-araATP, 2′F-araCTP, 2′F-araGTP and 2′F-araTTP catalyzed by DNA polymerases on a DNA template (PF33) (FIG. 10). Each of DV, 9N, Th and Ph polymerases can effectively incorporate all four 2′F-araNTPs to yield full length products while Bst, Taq, KF and MMLV polymerases generated prematurely terminated products.

### Primer Extensions with Mixtures of 2′F-araTTP and 2′F-araCTP

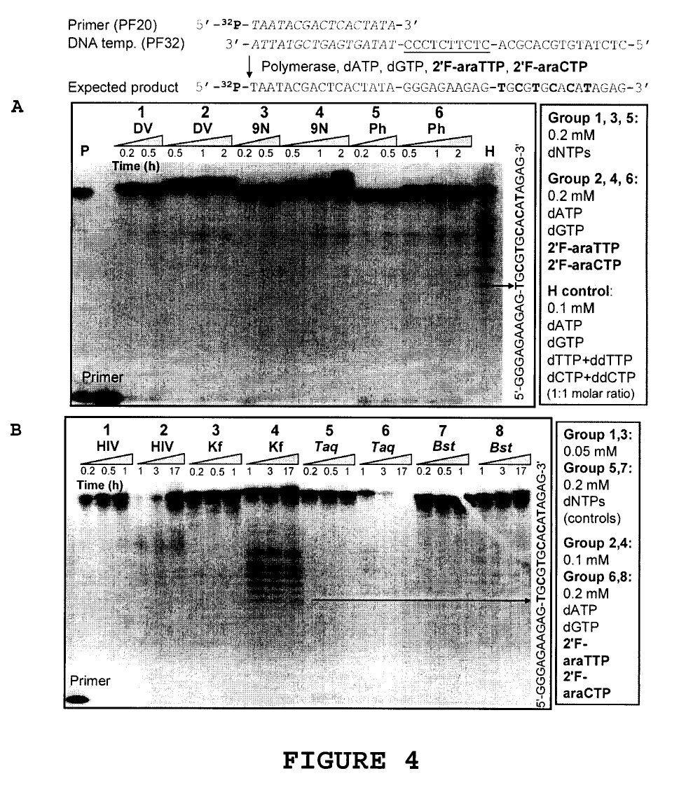

As shown in FIG. 4, primer extension also took place with a mixture of pyrimidine 2′F-araNTPs (2′F-araTTP and 2′F-araCTP) and purine dNTPs (dATP and dGTP). Under the conditions used, all the enzymes gave full-length products (blunt or overhang) with comparable efficiency as primer assays performed using the natural dNTPs (groups 1, 3, 5, 7; FIGS. 4A & B). No obvious pausing was observed for DV, 9N, Ph, Bst and Taq enzymes, in contrast to the results obtained with the Kf enzyme. Kf and HIV were able to incorporate 2′F-araNTP at lower concentrations (0.1 mM) compared to the thermophilic polymerases (0.2 mM). The high-fidelity enzyme Ph also provided full-length products, and the efficiency of polymerization was comparable to assays conducted only with the native dNTPs.

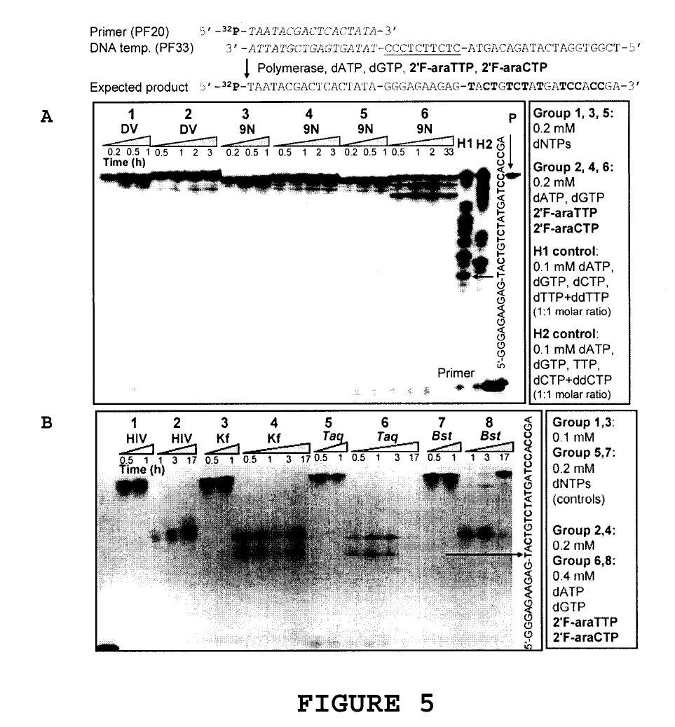

Ph incorporated twelve 2′F-araNTP units as easily as six units (FIG. 5), using the conditions described in Example 4. In this case, the “pausing” effect was observed for the last 2′F-araCTP incorporation, as assessed by comparison with ddNTP termination assays (lanes H1 and H2; FIG. 5A). HIV and Kf failed to give full-length products; under these conditions incorporation of only two 2′F-araNTP units occurred, followed by strong pausing (or termination) of DNA synthesis. In summary, multiple 2′F-araNTP units are more efficiently incorporated by thermophilic DNA polymerases, such as DV, 9N, Ph, Th and Bst DNA polymerases.

### DNA-FANA-template Directed Polymerizations

(a) DNA-FANA Template with 40% FANA Content

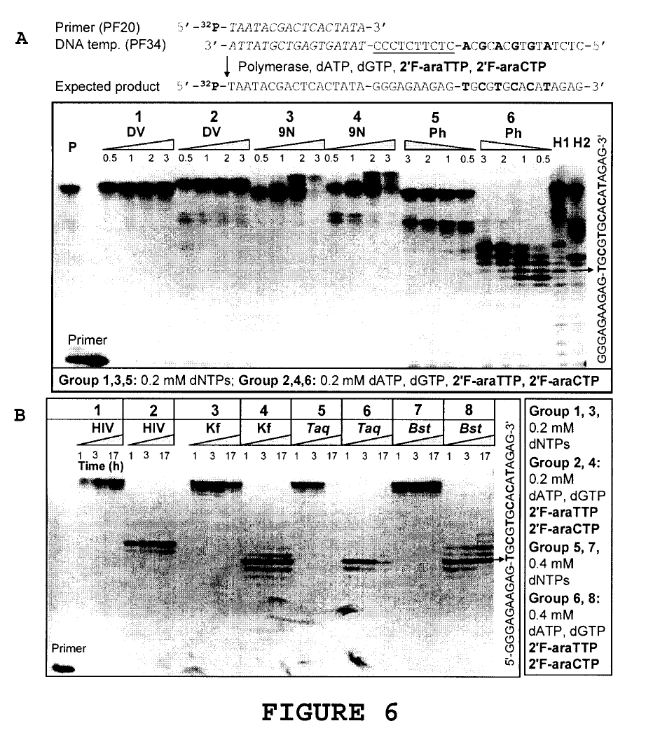

In the presence of only dNTPs, HIV, Kf, Taq, Bst, DV, 9N and Ph polymerases all recognized DNA-FANA (PF34) as a template to afford a full length DNA strand. With the exception of Ph DNA polymerase, little or no pausing was observed in these assays (FIGS. 6A & B).

Primer extension in the presence of 2′F-araNTP and dNTP was much more challenging; in fact, most of the enzymes tested (Ph, HIV, Kf, Taq and Bst) failed to provide the full-length products with the DNA-FANA PF34 template. The electrophoretic mobility of the products observed suggests that synthesis halted after incorporation of the first 2′F-araTTP unit (compare groups 2, 4 and 6, FIG. 6B to products formed in lanes H1 and H2; FIG. 6A). The notable exceptions were DV and 9N DNA polymerases. In these cases, efficient full-length product synthesis took place, with modest pausing observed after introduction of the last 2′F-araNTP (groups 2 and 4; FIG. 6A).

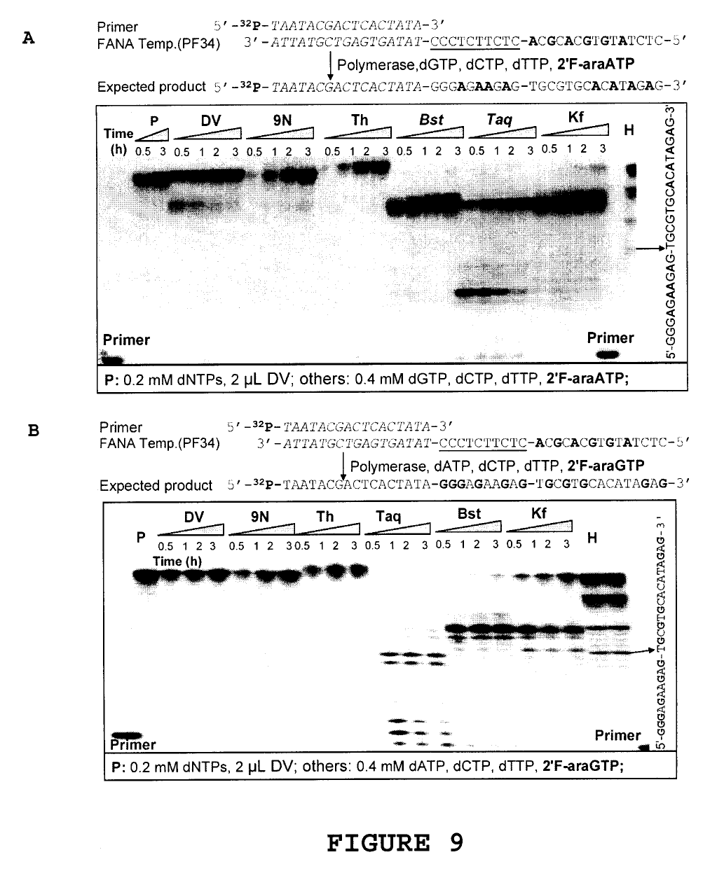

All of the enzymes studied can incorporate multiple 2′F-araATPs (FIG. 9A) and multiple 2′F-araGTPs (FIG. 9B) prior to the enzyme reaching a 2′F-araN unit on the template (within the running start sequence in the template PF34). Typically, the thermophilic enzymes DV and 9N were able to incorporate the 2′F-araATPs or 2′F-araGTPs on a FANA-DNA chimeric template much more efficiently than other polymerases (Bst, Taq, Kf. Th polymerase can also give full-length product as effectively as DV and 9N. Thus, multi 2′F-araA and 2′F-araG units can be incorporated by certain DNA polymerases (e.g. DV, 9N and Th) not only on a DNA template (i.e. segment 3′- . . . CCCTCTTCTC . . . -5′ of template PF34) but also on a chimeric DNA-FANA segment.

(b) DNA-FANA Template with 60% FANA Content

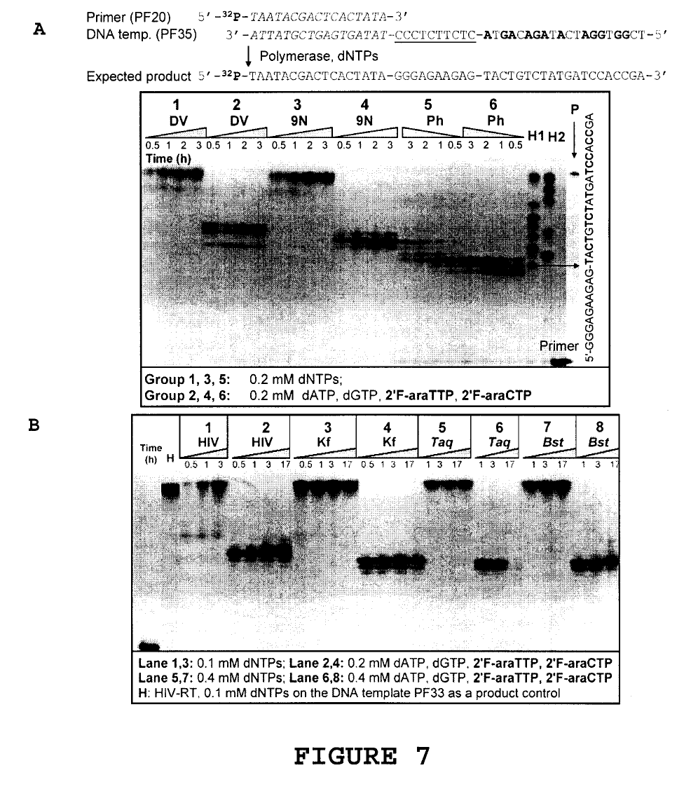

Next, DNA (and FANA-DNA) synthesis was assessed on the FANA-DNA template having a 60% 2′F-araN content (PF35, see Table 1). The experimental conditions and analyses described in Example 6 were followed, and representative results obtained are shown in FIG. 7.

With the exception of Ph polymerase, all enzymes tested (HIV, Kf, Taq, Bst, DV, 9N) efficiently utilized the 4 dNTPs and template PF34 to provide full-length DNA products (FIG. 7). In marked contrast, when dCTP and dTTP were replaced with the corresponding pyrimidine 2′F-araNTPs, all enzymes failed to produce full-length oligonucleotide product (FIG. 7). Consistent with Example 6, DV and 9N, incorporated 2′F-araNTP most efficiently (at least three 2′F-araNTPs) on this FANA template.

(c) All FANA-template (100% FANA content)

FANA was examined as a possible template for DNA synthesis with a variety of DNA polymerases (including reverse transcriptases) directing the incorporation of native dNTPs. To this end, template PF31, primer PF20 and all of the four natural dNTPs were incubated in the presence a DNA polymerase following the protocols and conditions described in Example 2 and Table 2.

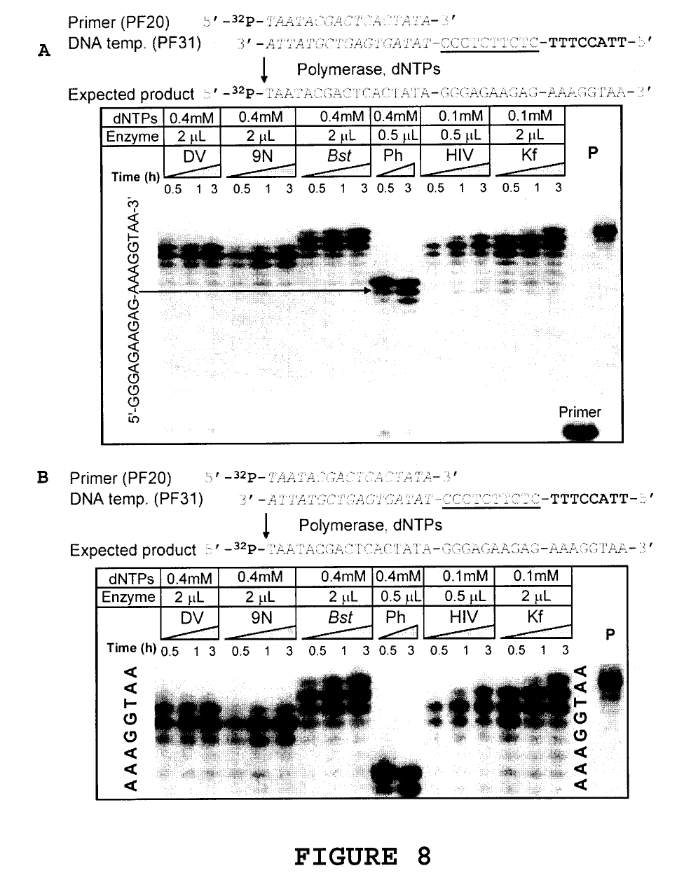

The data show that most enzymes can catalyze the extension of at least two dNTPs on the FANA template region. DV and 9N can incorporate up to 6 of the 8 dNTPs with main pausing after 5 nucleotides. HIV incorporated 7 nucleotides with main pausing after incorporation of 5-6 nucleotides, whereas Ph incorporated two dNTPs maximally. Remarkably, Kf and Bst afforded significant full-length products incorporating all eight dN residues on the FANA template (FIGS. 8 A&B).

### Fidelity of 2′F-araNTP Incorporation by DNA Polymerases

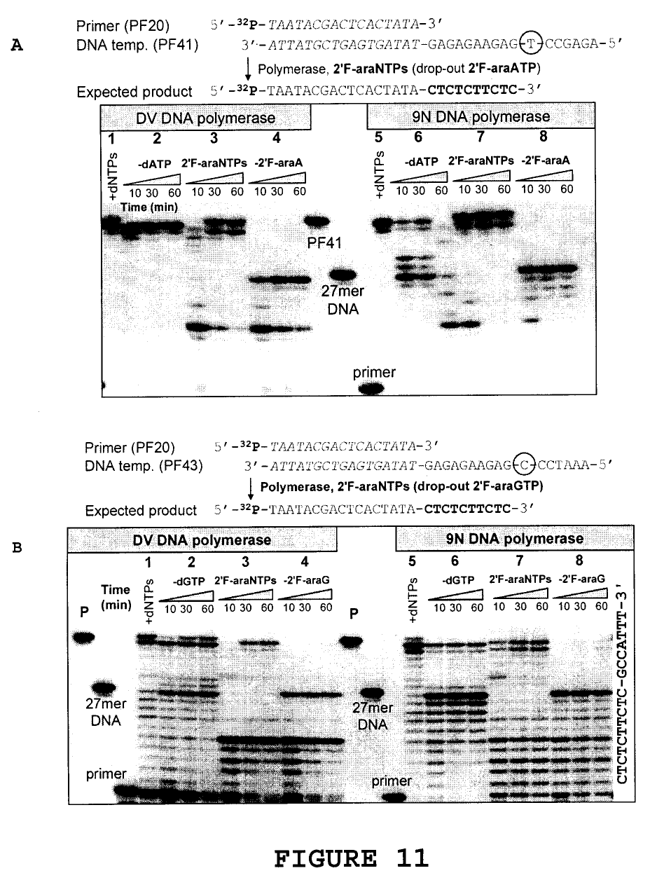

“Dropout assays” (Ichida et al. (2005) J. Am. Chem. Soc. 127:2802-2803) were conducted to assess the fidelity of 2′F-araNTP incorporation (each of 2′F-araATP, 2′F-araCTP, 2′F-araGTP and 2′F-araTTP) by various DNA polymerases (FIGS. 11-14). For example, the 2′F-araNTP of interest is removed from the pool, and the ensuing synthesis on DNA template (e.g. PF21, PF23, PF41, PF43) assessed in comparison with a control reaction containing all four 2′F-araNTPs. For comparison, dropout experiments containing dNTPs are run in parallel. Full-length DNA and FANA products are obtained when all four dNTPs (group 1 and 5) and 2′F-araNTPs (groups 3 and 7) were available, however, some pausing may occur during synthesis of the arabinose modified oligomers. Apparent fidelity was calculated according to the equation:

1−[(% full-length−2′F-araNTP)/(% full-length+2′F-araNTP)]

at 30 min reaction time (sample equation for dNTP values). >99% means that the full-length product was not detectable in our dropout assays.

### Comparative Primer Extension Efficacy of 2′F-araNTPs Versus 2′F-rNTPs

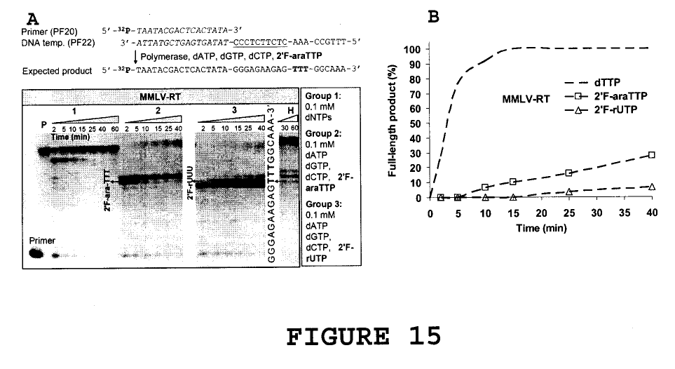

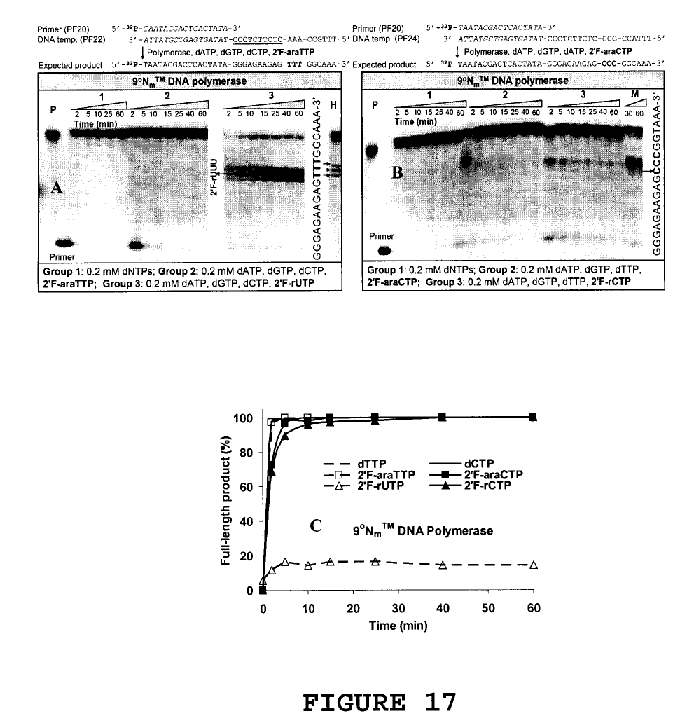

Primer extension assays were used to assess the incorporation efficiency of contiguous 2′F-ara(T/C)TPs compared to 2′F-r(U/C)TP on a DNA template (e.g. PF22 or PF24). To this end, a DNA template (e.g. PF22: FIGS. 15, 16, 17A, 18, 21A; PF24: FIGS. 17B, 19, 20, 21B), primer and the appropriate NTPs (e.g. combination of dATP/dCTP/dGTP/ and NTP, where N=dT, 2′F-araT, 2′F-rU, or rU (FIGS. 15, 16, 17A, 18, 21A); or combination of dATP/dGTP/dTTP/ and NTP, where N=dC, 2′F-araC, 2′F-rC, or rC (FIGS. 17B, 19, 20, 21B)), were incubated in the presence one of MMLV-RT, 9N, HIV or DV polymerase. The protocols and conditions are described in Example 2 and Table 2.

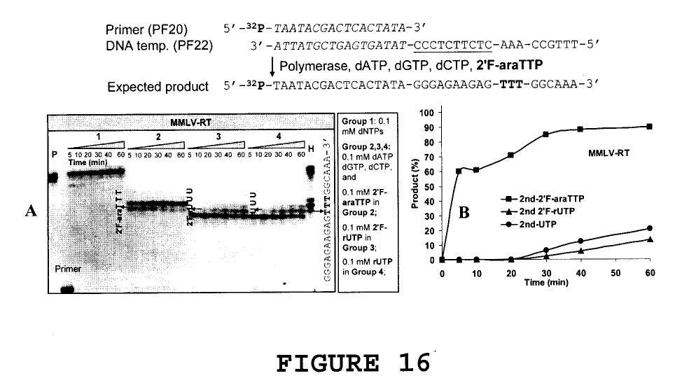

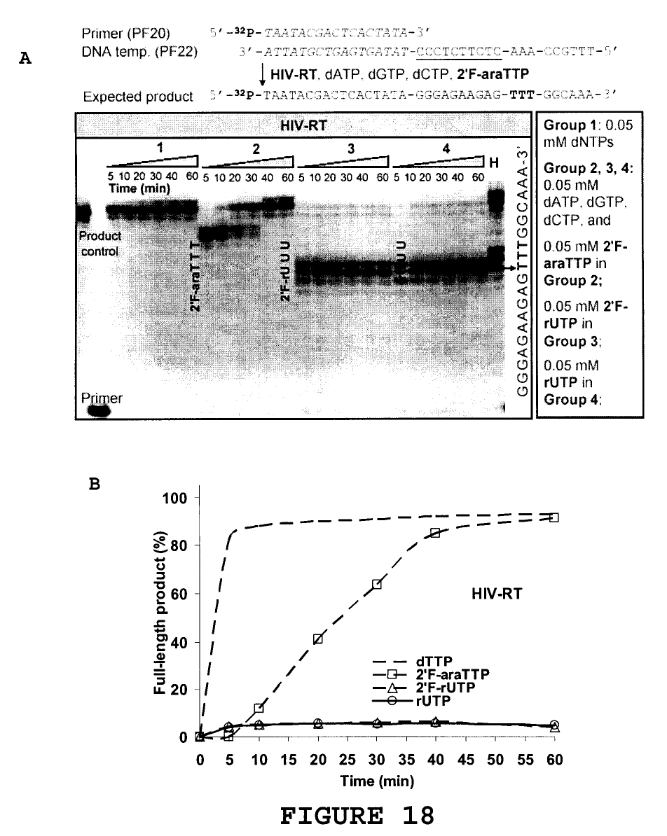

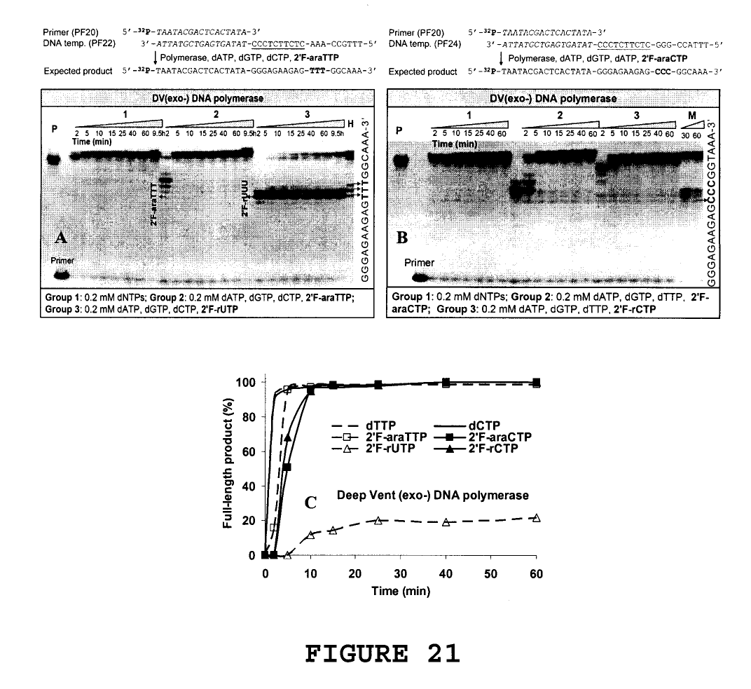

Under these conditions, 2′F-araTTP was an excellent substrate of HIV (FIG. 18), and its incorporation proceeded more efficiently compared to 2′F-rUTP and rUTP. Different pausing patterns were observed depending on the combination of NTPs used in the assay. Delayed pausing was observed after the last 2′F-araTTP was incorporated, whereas for the rNTPs (e.g. 2′F-rUTP or rUTP), strong pausing occurred prior or immediately after incorporation of the first rNTP (FIG. 18). Similar results were obtained with MMLV. This enzyme was able to incorporate 2′F-araTTP more efficiently than 2′F-rUTP and rUTP (FIGS. 15 & 16). Similarly, pausing was observed after the first incorporation of 2′F-araTTP, and before the first incorporation of 2′F-rUTP and rUTP. By analyzing the second incorporations of modified triphosphates in FIG. 16, it is clear that 2′F-araTTP is the best substrate of MMLV. With thermostable polymerases DV, incorporation of 2′F-araTTPs proceeded efficiently giving after 5 min nearly quantitative yield of full length product (FIG. 21A). Accumulation of an intermediate band corresponding to 5′-[DNA primer]-2′F-ara(TpTpT)-dG-3′ was observed in the first 2 min. With 2′F-rUTP, however, abrupt pausing was observed after the first and second 2′F-rUTP incorporation, yielding only 20% of the desired full-length product after 9.5 h (FIG. 21A). The same trends were observed with 9N DNA polymerase (FIG. 17A). Incorporation efficiencies of 2′F-araTTP and dTTP were comparable and ca.100% full-length products were generated within 2 min and without pausing, whereas the reaction with 2′F-rUTP paused after one incorporation producing little of the expected full-length product (ca. 20% after one hour).

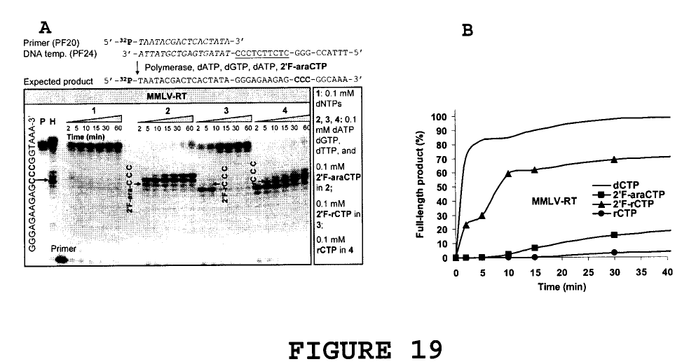

Using similar primer extension assays in combination with the DNA template PF24, incorporation efficiency was shown to follow the order of: dCTP=2′F-rCTP>2′F-araCTP>>rCTP with significant amounts of full length products observed for both 2′F-araCTP and 2′F-rCTP incorporation (FIGS. 17B, 19, 20 & 21B). For HIV and MMLV-RT. The incorporation of 2′F modified CTPs seemed to be more complicated than what was previously shown with 2′F modified T (U)TPs (FIGS. 16, 17B, 18, 21A). Generally, the incorporation efficiency of 2′F-araCTP by HIV and MMLV is comparable to or slightly less efficient than that observed with 2′F-rCTP but better than rCTP. Remarkably, 2′F-rCTP behaved very differently from rCTP, e.g, little or no pausing was observed with these enzymes. On the other hand, assays with rCTP and either HIV and MMLV showed significant pausing before the first rCTP incorporation, as observed earlier with rUTP (FIGS. 19 & 20). DV and 9N were able to incorporate 2′F-araCTP as well as 2′F-rCTP under the conditions studied here (FIGS. 17B and 21B).

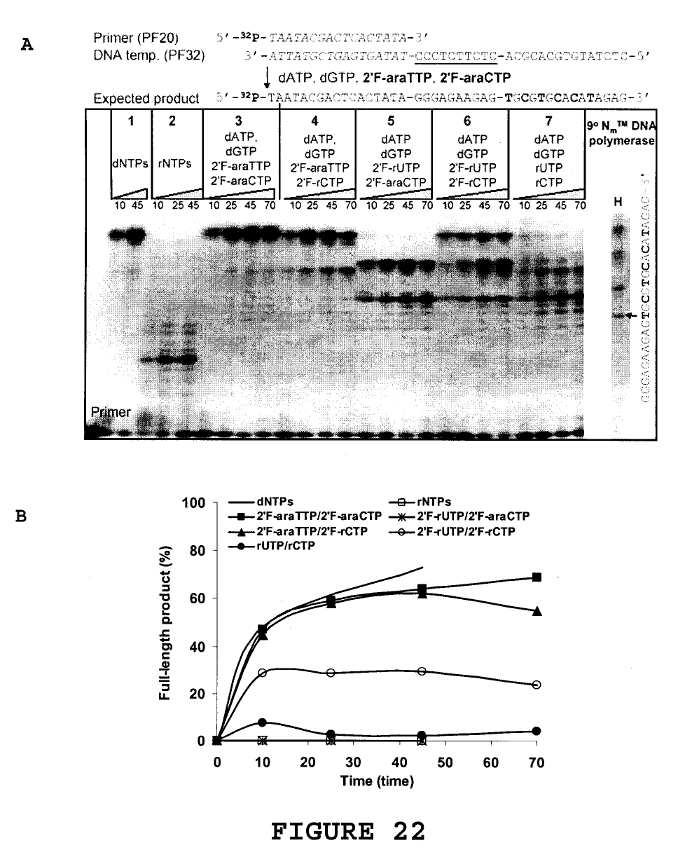

The methods described also provide unexpected examples of chimeric oligonucleotides containing both 2′F-ara and another modified nucleotide. Polymerization is carried out using a ³²P-labelled primer/DNA template (e.g. PF32) pair, and various NTP combinations following the protocol described in Example 2 and Table 2. More specifically, the primer/template pair (e.g. PF20/PF32) was incubated in the presence of 9N DNA polymerase, dATP, dGTP and one of four pyrimidine pairs: (a) 2′F-araTTP/2′F-araCTP; (b) 2′F-araTTP/2′F-rCTP; (c) 2′F-rUTP/2′F-rCTP; and (d) rUTP/rCTP. As shown in FIG. 22 and Table 5, 9N polymerase can incorporate 2′F-araTTP/2′F-araCTP and 2′F-araTTP/2′F-rCTP much more efficiently than 2′F-rUTP/2′F-rCTP, 2′F-rUTP/2′F-araCTP, and rUTP/rCTP under these conditions, exemplifying 2′-fluoro-ribonucleoside-5′triphosphate (2′F-RNA) (FIG. 22). Other modified nucleotides could include ribonucleoside 5′-(alpha-P-borano)-triphosphates (BH₃-RNA), ribonucleoside 5′-(alpha-thio)triphosphates (S-RNA), 2′-deoxyribonucleoside 5′-(alpha-methyl)triphosphates (P-Me DNA), alpha-L-threofuranosyl nucleoside 5′-triphosphates (TNA), 4′-thio-ribonucleoside 5′-triphosphates (4′S-RNA), 2′-amino-ribonucleoside-5′triphosphate (2′NH₂-RNA), 2′-deoxy-2′-fluororibonucleoside-5′-triphosphate (2′F-DNA).

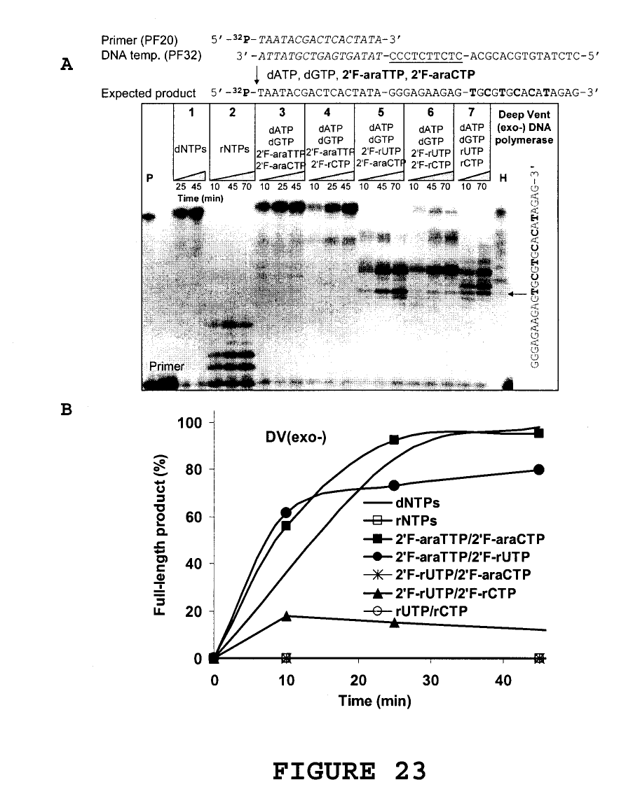

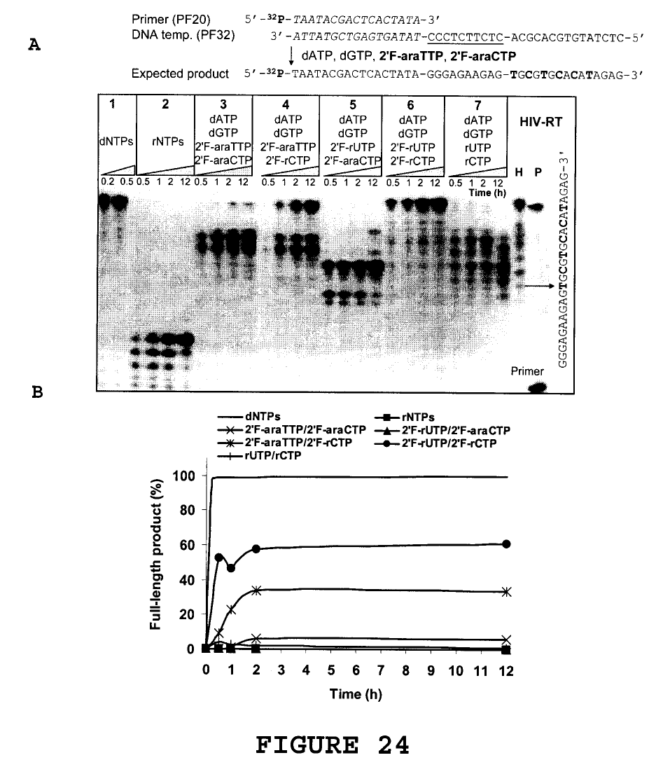

Polymerization experiments were also conducted using DV and HIV polymerases (FIGS. 23 and 24 respectively).

In summary, in contrast to previous experiments showing that 2′F-araNTPs are potent inhibitors of DNA polymerases and/or terminators of DNA synthesis (see Background of Invention), the enzymatic conditions described herein with 2′F-araNTPs promote efficient DNA-FANA synthesis on DNA, DNA-FANA and FANA templates. As summarized in Table 3, all enzymes tested were able to incorporate at least one 2′F-araNTP nucleotide when the template was DNA or a chimeric DNA-FANA strand. The DNA polymerases Bst, DV and 9N were the most capable of catalyzing multiple 2′F-araNTP additions to the DNA primer. The results described below also demonstrate that it is possible to carry out both DNA and DNA-FANA synthesis on templates containing a large proportion of 2′F-araN residues (up to 40%), although it was not possible to synthesize DNA-FANA strands on templates constructed with a very large 2′F-araN content (e.g. 60%). Among the enzymes tested Bst and KL are unique in catalyzing the incorporation of either successive dNTP residues on an all FANA template. The methods described herein also allow for the synthesis of oligonucleotide analogues containing both 2′F-ribonucleotides and 2′F-arabinonucleotides. This opens the possibility of synthesizing, via SELEX, a large number of oligonucleotides containing 2′F-araN and any other modified nucleodie that is a substrate of DNA polymerases (e.g. TNA and FANA). It is also understood that further chemical modifications can be introduced, via post-SELEX synthesis, to further enhance the pharmacokinetics and nuclease stability of a nucleic acid aptamer/ligand.

The following examples are illustrative of various aspects of the invention, and do not limit the broad aspects of the invention as disclosed herein.

### Example 1

### Materials and Methods

### A. DNA and FANA Templates and Primer Design

DNA and FANA-DNA templates and primers used are shown in Table 1. The primer (PF20) is a 17 nt DNA sequence 5′-taatacgactcactata-3′. Each template strand comprises three sequence segments (see Table 1): a 17 nt long primer binding sequence 5′-tatagtgagtcgtatta-3′, a 10 nt long running start sequence 5′-ctcttctccc-3′, and a variable “test sequences” made up of an all-DNA or chimeric DNA-FANA segment; these test sequences include DNA segments (PF22, PF24, PF30, PF32, PF33) which are designed to test the incorporation of three 2′F-araT units (PF22), three 2′F-araC units (PF24), six mixed 2′F-araT and 2′F-araC units (PF32), and 12 mixed 2′F- and 2′F-araC units (PF33); they also include DNA-FANA chimeras, i.e., PF34 (40% 2′F-araN content), PF35 (60% 2′F-araN content) and PF31 (100% 2′F-araN content). All DNA sequences were obtained from commercial sources (University of Calgary DNA Services). Oligonucleotide FANA-DNA templates were synthesized according to the published procedures [Elzagheid, M. et al. Viazovkina, E. et al. (2002) In Current Protocols in Nucleic Acid Chemistry, Vol. 4.15. John Wiley & Sons, pp. 1-22.]

TABLE 1
Base sequence of oligonucleotide templates and
primer

| | | | | SEQ ID |
|---|---|---|---|---|
| Code | Type | Sequence* | N-mer | NO. |
| PF20 | Primer | 5′-TAATACGACTCACTATA-3′ | 17 | 1 |
| PF22 | DNA | 5′-TTTGCCAAA-CTCTTCTCCC- | 36 | 2 |
| | temp. | TATAGTGAGTCGTATTA-3′ | | |
| PF24 | DNA | 5′-TTTACCGGG-CTCTTCTCCC- | 36 | 3 |
| | temp. | TATAGTGAGTCGTATTA-3′ | | |
| PF30 | DNA | 5′-TTACCTTT-CTCTTCTCCC- | 35 | 4 |
| | temp. | TATAGTGAGTCGTATTA-3′ | | |
| PF31 | FANA- | 5′-TTACCTTT-CTCTTCTCCC- | 35 | 5 |
| | DNA | TATAGTGAGTCGTATTA-3′ | | |
| | temp. | | | |
| PF32 | DNA | 5′-CTCTATGTGCACGCA- | 42 | 6 |
| | temp. | CTCTTCTCCC- | | |
| | | TATAGTGAGTCGTATTA-3′ | | |
| PF33 | DNA | 5′-TCGGTGGATCATAGACAGTA- | 47 | 7 |
| | temp. | CTCTTCTCCC- | | |
| | | TATAGTGAGTCGTATTA-3′ | | |
| PF34 | FANA- | 5′-CTCTATGTGCACGCA- | 42 | 8 |
| | DNA | CTCTTCTCCC- | | |
| | temp. | TATAGTGAGTCGTATTA-3′ | | |
| PF35 | FANA- | 5′-TCGGTGGATCATAGACAGTA- | 47 | 9 |
| | DNA | CTCTTCTCCC- | | |
| | temp. | TATAGTGAGTCGTATTA-3′ | | |
| PF41 | DNA | 5′-AGAGCCTGAGAAGAGAG- | 34 | 10 |
| | temp. | TATAGTGAGTCGTATTA-3′ | | |
| PF43 | DNA | 5′-AAATCC-C-GAGAAGAGAG- | 34 | 11 |
| | temp. | TATAGTGAGTCGTATTA-3′ | | |
| PF21 | DNA | 5′-TTTGCC-A-CTCTTCTCCC- | 34 | 12 |
| | temp. | TATAGTGAGTCGTATTA-3′ | | |
| PF23 | DNA | 5′-TTTACC-G-CTCTTCTCCC- | 34 | 13 |
| | temp. | TATAGTGAGTCCTATTA-3′ | | |
| PF39 | DNA | 5′-TAATACGACTCACTA | 27 | 14 |
| | temp. | TAGGGAGAAGAG-3′ | | |

*Note: Bold and capital letter: 2′F-araN units; Capital letters: DNA; Italicized sequence: primer binding sequence and primer (17 nt); Underlined sequence: a running start (10 nt).
B. Source of Enzymes and Nucleoside 5′-triphosphates

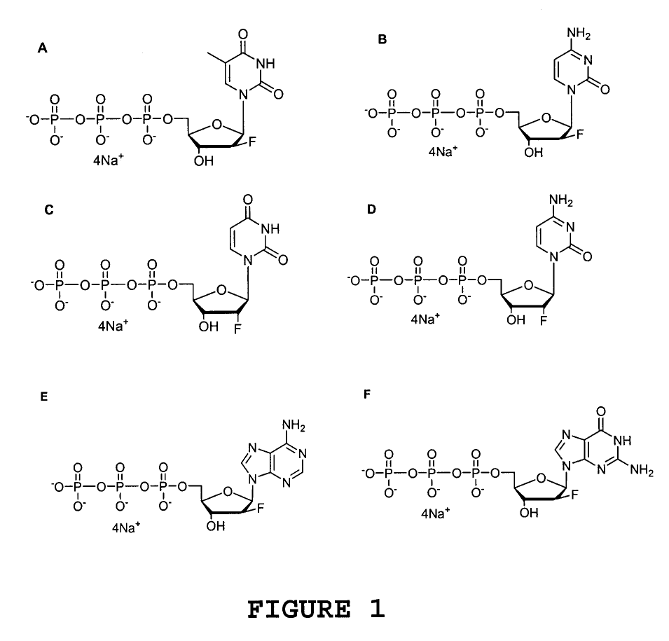

2′-Deoxyribonucleoside and ribonucleoside 5′-triphosphates (dNTPs and rNTPs) were purchased from Fermentas. 2′F-araT and 2′F-araC were synthesized by published procedures. The introduction of the 5′-triphosphate moiety was conducted by Rasayan, Inc. (Encinitas, Calif., USA). The structures of the fluorinated nucleoside 5′-triphosphates are shown in FIG. 1. DNA polymerase enzymes were as follows:

(a) Thermophilic DNA polymerases (source: New England Biolabs):
Deep Vent® (3′→5′ exo-) DNA polymerase (DV);
9° Nm™ DNA polymerase (9N);
Therminator™ DNA polymerase (Th)
Bst DNA polymerase large fragment (Bst);
Taq DNA polymerase (Taq);
Phusion™ High-Fidelity DNA polymerase (Ph).
   (b) Mesophilic DNA polymerase (source: New England Biolabs):
Klenow fragment DNA polymerase (3′ 5′ exo-) (KF)
   (c) Reverse transcriptases (RT):
MMLV-RT (MMLV or MM; source: Fermentas);
HIV-1 RT (recombinant) (HIV; source: Worthington Biochemical Corp.).

### C. Conditions for DNA Polymerization Reactions

Unless otherwise noticed in the Examples described below, the conditions used in the primer extension assays, such as dNTP concentration, reaction temperatures, concentration of Mg²⁺, and reaction buffers (provided by the manufacturers, except for the HIV buffer which was prepared in-house) are shown in Table 2.

TABLE 2
Polymerase assay conditions for primer extensions

| | | | | |
|---|---|---|---|---|
| | Activity | | | |
| | (units/ | | Temp | |
| Enzyme | μL) | [dNTP] | used | Reaction buffer (x1) |

Thermophilic DNA polymerases

| | | | | |
|---|---|---|---|---|
| Deep | 2 | 0.2 mM | 55° C. | 20 mM Tris-HCl (pH 8.8 |
| Vent ® | | | | at 25° C.), 10 mM |
| (exo-) | | | | (NH₄)₂SO₄, 10 mM KCl, |
| | | | | 2 mM MgSO₄, 0.1% |
| | | | | Triton X-100, |
| 9°Nₘ ™ | 2 | 0.2 mM | 55° C. | Same as above |
| Bst | 8 | 0.2 mM | 55° C. | Same as above |
| Th | 2 | 0.2 mM | 55° C. | Same as above |
| Taq | 5 | 0.2 mM | 55° C. | Same as above |
| Phusion ™ | 5 | 0.2 mM | 55° C. | 5X Phusion ™ HF |
| | | | | Buffer |

Mesophilic DNA polymerases

| | | | | |
|---|---|---|---|---|
| Klenow | 5 | 0.033 mM | 37° C. | 10 mM Tris-HCl (pH 7.5 |
| (exo-) | | | | at 25° C.), 5 mM MgCl₂, |
| | | | | 7.5 mM DTT |

Reverse transcriptases

| | | | | |
|---|---|---|---|---|
| MMLV-RT | 20 | 0.5 mM | 37° C. | 50 mM Tris-HCl (pH 8.3 |
| | | | | at 25° C.), 50 mM KCl, |
| | | | | 4 mM MgCl₂, 10 mM |
| | | | | DTT. |
| HIV-1 RT | 27.3 | 0.2 mM | 37° C. | 50 mM Tris-HCl |
| | | | | (pH 7.8), 60 mM KCl, |
| | | | | 2.5 mM MgCl₂ |

### Example 2

### Incorporation of Three Contiguous 2′F-araT Units

Primer extension assays were used to assess the incorporation of 2′F-araTTP on the DNA template (PF22). The DNA primer (PF20) was first radioactively labeled at the 5′-hydroxyl terminus with a radioactive phosphorus probe and the enzyme T4 polynucleotide kinase (T4 PNK) according to the manufacturer's specifications (MBI Fermentas Life Sciences, Burlington, ON). Incorporation of the ³²P label was accomplished in reaction mixtures consisting of DNA substrate (200 pmol), 2 μl loxreaction buffer (Buffer A for forward reaction: 500 mM Tris-HCl, pH 7.6 at 25° C., 100 mM MgCl₂, 50 mM DTT, 1 mM spermidine and 1 mM EDTA), 1 μl T4 PNK enzyme solution (10 U/1 μl in a solution of 20 mM Tris-HCl, pH 7.5, 25 mM KCl, 0.1 mM EDTA, 2 mM DTT and 50% glycerol), 6 μl [γ-³²P]-ATP solution (6000 Ci/mmol, 10 mCi/ml; Amersham Biosciences, Inc.) and autoclaved sterile water to a final volume of 20 μl. The reaction mixture was incubated for about 45-60 min at 37° C., followed by a second incubation for 10 min at 95° C. to heat denature and deactivate the kinase enzyme. The solution was purified according to a standard protocol and the isolated yield of 5′-³²P-DNA following gel extraction averages 50%. The pure labeled samples were kept at −20° C. for future use.

Usually 2 or 3 folds of cold (unlabeled) DNA primer were combined with the radioactive primer. Then primer to template was mixed together with the final concentration at 85 nM for the primer and 255 nM for the template (i.e. the primer:template=1:3 molar ratio). The primer and template were heated at 95° C. for 5 min and annealed at 4° C. for at least two hours before use. In an Eppendorf tube, the following reagents, 5× or 10× buffer, nucleoside 5′-triphosphates (DATP, dGTP, dCTP, 2′F-araTTP at the same concentrations; see legend of FIG. 2), water, the primer and template mixture, were added together according to pre-calculated reaction volume (20 μL) and concentrations (see reaction conditions in Table 2). Mineral oil (20 μL) was usually used to prevent evaporation. The reaction mixture was incubated at either 37° C. or 55° C. and one of the various enzymes tested was added to initiate the primer extension reactions. Time points were obtained by taking 4 μL or 8 μL aliquot from the reaction mixture and quenching the aliquot by the same volume of a stopping dye solution (98% deionized formamide, 10 mM EDTA, 1 mg/ml bromophenol blue and 1 mg/ml xylene cyanol). The product pattern from each timepoint was analyzed by 12% denaturing polyacryamide gel electrophoresis (PAGE) and subsequent autoradiography.

Under these conditions, all of the enzymes conditions gave full-length products, although to different extents (FIG. 2). Pausing after incorporation of the first 2′F-araTTP unit was observed, as indicated by the mobility of the marker bands formed in the chain termination assays with ddTTP. Remarkably, the thermophilic enzymes DV, 9N and Taq were able to incorporate 2′F-araTTP units even at 37° C., and the high-fidelity Ph could also afford full-length products. Furthermore, the polymerase KF was able to incorporate 2′F-araTTP at a lower concentration (0.033 mM). These results show that it is possible to incorporate three consecutive 2′F-araTTP units on the primer strand.

### Example 3

### Incorporation of Three Contiguous 2′F-araC Units

Primer extension assays were used to assess the incorporation of 2′F-araCTP on the DNA template (PF22). The DNA primer (PF20) was first radioactively labeled at the 5′-hydroxyl terminus with a radioactive phosphorus probe (³²P) Usually 2 or 3 folds of cold DNA primer were combined with the radioactive primer. Then primer to template was mixed together with the final concentration at 85 nM for the primer and 255 nM for the template (i.e. the primer:template=1:3 molar ratio). The primer and template were heated at 95° C. for 5 min and annealed at 4° C. for at least two hours before use. In an Eppendorf tube, the following reagents, 5× or 10× buffer, triphosphates (dATP, dGTP, dTTP, 2′F-araCTP at the same concentrations; see legend of FIG. 3), water, the primer and template mixture, were added together according to pre-calculated reaction volume (20-40 μL) and concentrations (see reaction conditions in Table 2). Mineral oil (20 μL) was usually used to prevent evaporation during the course of the reactions. The reaction mixture was incubated at either 37° C. or 55° C. and different enzyme was added to initiate the primer extension reaction. Time points were obtained by taking 4 μL or 8 μL aliquot from the reaction mixture and quenching the aliquot by the same volume of stopping dye solution (98% deionized formamide, 10 mM EDTA, 1 mg/ml bromophenol blue and 1 mg/ml xylene cyanol). The product pattern from each time point was analyzed by 12% denaturing polyacryamide gel electrophoresis (PAGE) and subsequent autoradiography.

As shown in FIG. 3, the mesophilic and retroviral enzymes gave full-length products, with some pausing detected after incorporation of the first 2′F-araC unit (37° C.). Under the same conditions (37° C.), the thermophilic enzymes DV and Taq incorporated three consecutive 2′F-araCTP units, but 9N and Bst could not. However, 9N and Bst were capable of readily incorporating one 2′F-araC unit as easily as one 2′F-araT unit (data not shown). At a higher temperature (55° C.), 9N and Bst were able to incorporate three 2′F-araC units and gave the expected full-length products (FIG. 3). The high-fidelity enzyme, Ph, also provided a full-length product. Incorporation efficiencies of DNA polymerases for three 2′F-araT(C)TPs are given in Table 3.

TABLE 3
Polymerase used and incorporation efficiency of three 2′F-araT(C)TPs.

| | | | |
|---|---|---|---|
| | | Incorporation | Incorporation |
| | Short | of three | of three |
| Polymerases * | name | 2′F-araTTP | 2′F-araCTP |

Thermophilic DNA polymerases (assays conducted at
55° C. except otherwise noticed)

| Deep Vent (3′→5′ exo-) | DV | ++++ | (37° C.) | +++ | (37° C.) |
|---|---|---|---|---|---|
| DNA polymerase | | | | | |
| | | | | ++++ | |
| 9° Nₘ ™ DNA polymerase | 9N | ++++ | (37° C.) | None | (37° C.) |
| | | | | ++++ | |
| Bst DNA polymerase large | Bst | ++ | (37° C.) | None | (37° C.) |
| fragment | | | | | |
| | | +++ | | ++++ | |
| Taq DNA polymerase | Taq | ++ | (37° C.) | ++ | (37° C.) |

| | | | ++++ |
|---|---|---|---|
| Phusion ™ High-Fidelity | Ph | ++++ | ++++ |
| DNA polymerase | | | |

Mesophilic DNA polymerase (assays conducted at 37° C.)

| | | | |
|---|---|---|---|
| Klenow fragment DNA | Kf | ++ | ++ |
| polymerase (3′→ 5′ exo-) | | | |

Reverse transcriptase DNA polymerases (assays conducted at 37° C)

| | | | |
|---|---|---|---|
| HIV-1 RT (recombinant) | HIV | ++++ | ++++ |
| Moloney Murine Leukemia | MMLV | +++ | ++++ |
| Virus RT | or MM | | |

* Enzyme sources: all from New England Biolabs (NEB) except for Ph from Finnzymes (distributed by NEB); HIV-1 RT from Worthington Biochemical Corp.;

### Example 4

### Incorporation of Six 2′F-araT/C Units

Again, primer extension assays were used to assess the incorporation of three 2′F-araTTPs and three 2′F-araCTTPs on the DNA template (PF32). The DNA primer (PF20) was first radioactively labeled at the 5′-hydroxyl terminus with a radioactive phosphorus probe (³²P). Usually 2 or 3 folds of cold DNA primer were combined with the radioactive primer. The primer and template strands were mixed together in a 1:3 molar ratio (final concentration: 85 nM primer: 255 nM template), heated at 95° C. for 5 min, and annealed at 4° C. for at least two hours before use. In an Eppendorf tube, equimolar concentrations nucleoside 5′-triphosphates (DATP, dGTP, 2′F-araTTP, 2′F-araCTP) were combined with the primer and template mixture according to the conditions listed on Table 2 and FIG. 4. Mineral oil (20 μL) was usually used to prevent evaporation during reactions conducted a high temperatures (55° C.). The reaction mixture was incubated at either 37° C. or 55° C. and one of the various enzymes tested was added to initiate the primer extension reaction. Time points were obtained by taking 4 μL or 8 μL aliquot from the reaction mixture and quenching the aliquot by the same volume of stopping dye solution (98% deionized formamide, 10 mM EDTA, 1 mg/ml bromophenol blue and 1 mg/ml xylene cyanol). The product pattern from each time point was analyzed by 12% denaturing polyacryamide gel electrophoresis (PAGE) and subsequent autoradiography.

Under these conditions, all the enzymes gave full-length products (blunt or overhang) with comparable efficiency as primer assays performed using the natural dNTPs (FIG. 4). No obvious pausing was observed for DV, 9N, Ph, Bst and Taq enzymes, in contrast to the results obtained with the Kf enzyme. Kf and HIV were able to incorporate 2′F-araTTP at lower concentrations (0.1 mM) compared to the thermophilic polymerases (0.2 mM). High-fidelity Ph also gave the full-length product, and the efficiency of polymerization was comparable to assays conducted only with the native dNTPs. These results show that primer extension can take place with mixtures of pyrimidine 2′F-araNTPs.

### Example 5

### Incorporation of Twelve 2′F-araT/C Units

Primer extension assays were used to assess the incorporation of twelve 2′F-araNTP units (6×2′F-araTTPs+6×2′F-araCTPs) on the DNA template PF33. Two-three folds of cold DNA primer (PF20) was combined with the ³²P-labeled DNA primer (PF20), and mixed together with the DNA template strand PF33 in a 1:3 molar ratio (final concentration: 85 nM primer: 255 nM template). The resulting mixture was heated at 95° C. for 5 min, and annealed at 4° C. for at least two hours prior to use. In an Eppendorf tube, equimolar concentrations nucleoside 5′-triphosphates (DATP, dGTP, 2′F-araTTP, 2′F-araCTP) were combined with the primer and template mixture according to the conditions listed on Table 2 and FIG. 5. Mineral oil (20 μL) was usually used to prevent evaporation. The reaction mixture was incubated at either 37° C. or 55° C. and different enzyme was added to initiate the primer extension reaction. Time points were obtained by small aliquots (4 μL or 8 μL) from the reaction mixture and quenching the aliquot with the same volume of a stopping dye solution (98% deionized formamide, 10 mM EDTA, 1 mg/ml bromophenol blue and 1 mg/ml xylene cyanol). The product pattern from each time point was analyzed by 12% denaturing polyacryamide gel electrophoresis (PAGE) and subsequent autoradiography.

Under these conditions, thermophilic enzymes (DV, 9N, Ph and Bst) incorporated the 2′F-araNTPs more efficiently compared to the HIV and KF polymerases (FIG. 5). For some reason, degradation of the primer took place for Taq DNA polymerase reaction assay, and only shorter intermediate products were observed. In order to drive the complete incorporation of the two 2′F-araNTPs, double amount of enzyme were used except for high-fidelity Ph. In fact, Ph incorporated twelve 2′F-araNTP units as easily as six units, using the conditions described in Example 4. The pausing effect was only observed for the last 2′F-araCTP incorporation, as assessed by comparison with ddNTP termination assays (lanes H1 and H2; FIG. 5A). HIV and KF failed to give full-length products; under these conditions incorporation of only two 2′F-araNTP units occurred, followed by strong pausing (or termination) of DNA synthesis. In summary, multiple 2′F-araNTP units are shown to be incorporated by thermophilic DNA polymerases, such as DV, 9N, Ph, and Bst DNA polymerases.

### Example 6

### FANA-DNA Template-mediated Oligonucleotide Synthesis

### Assay 1

Unlike the methods described above which involve DNA as the template strand (Examples 2-5), the following primer extension assay uses a chimeric FANA-DNA strand having 40% 2′F-araN content in the testing sequence segment (Table 1). Cold DNA primer (PF20) was combined with the ³²P-labeled DNA primer (PF20), and mixed together with the FANA-DNA template strand PF34 (FIG. 6) in a 1:3 molar ratio (final concentration: 85 nM primer: 255 nM template). The resulting mixture was heated at 95° C. for 5 min, and annealed at 4° C. for at least two hours prior to use. In an Eppendorf tube, nucleoside 5′-triphosphates (i.e., either the four dNTPs, or an equimolar mixture of DATP, dGTP, 2′F-araTTP, 2′F-araCTP) were combined with the primer/template (see Table 2 and FIG. 6 for conditions). The reaction mixture was incubated at either 37° C. or 55° C. and one of the various enzymes tested was added to initiate the primer extension reaction. A small aliquot (4-8 μL) was removed from the reaction mixture at various time intervals, and analyzed via gel electrophoresis/autoradiography after quenching with stopping dye solution (98% deionized formamide, 10 mM EDTA, 1 mg/ml bromophenol blue and 1 mg/ml xylene cyanol).

In the presence of only dNTPs, HIV, Kf, Taq, Bst, DV, 9N and Ph DNA polymerases all recognized PF34 as a template to afford full length all-DNA products. With the exception of Ph, little or no pausing was observed (FIG. 6). For some reason, degradation of the primer took place for Taq DNA polymerase reaction assay, and only shorter intermediate products were observed.

Incorporation of 2′F-araNTPs was much more challenging; in fact, most of the enzyme tested (Ph, HIV, Kf, Taq and Bst) failed to provide the full-length products with the PF34 template. The electrophoretic mobility of the products observed suggests that synthesis halted after incorporation of the first 2′F-araTTP unit (FIG. 6). The notable exceptions were DV and 9N DNA polymerases. In these cases, efficient full-length product synthesis took place, with modest pausing observed after introduction of the last 2′F-araNTP.

### Example 7

### FANA/DNA Template-mediated Oligonucleotide Synthesis

### Assay 2

Next, DNA (and FANA-DNA) synthesis was assessed on the FANA-DNA template having a 60% 2′F-araN content (PF35, see Table 1). The experimental conditions and analyses described in Example 6 were followed, and representative results obtained are shown in FIG. 7.

With the exception of Phusion polymerase, all enzymes tested (HIV, Kf, Taq, Bst, DV, 9N) efficiently utilized the 4 dNTPs and template PF34 to provide full-length DNA products (FIG. 7). In marked contrast, when dCTP and dTTP were replaced with the corresponding pyrimidine 2′F-araNTPs, all enzymes failed to produce full-length oligonucleotide product (FIG. 7). Consistent with Example 6, DV and 9N, incorporated 2′F-araNTP most efficiently (at least three 2′F-araNTPs) on this FANA template.

### Example 8

### FANA as a Template for Polymerase-directed DNA Synthesis

FANA was examined as a possible template for DNA synthesis with a variety of DNA polymerases (including reverse transcriptases) directing the incorporation of native dNTPs. To this end, template PF31, primer PF20 and all of the four natural dNTPs were incubated in the presence a DNA polymerase following the protocols and conditions described in Example 2 and Table 2.

The data shows that most enzymes can catalyze the extension of at least two dNTPs on the FANA template region. DV and 9N can incorporate up to 6 of the 8 dNTPs with main pausing after 5 nucleotides. HIV incorporated 7 nucleotides with main pausing after incorporation of 5-6 nucleotides, whereas Ph incorporated two dNTPs maximally. Remarkably, Kf and Bst afforded significant full-length products incorporating all eight dN residues on the FANA template (FIGS. 8 A&B).

### Example 9

### Incorporation of Multi 2′F-araA or 2′F-araGTP Units

Primer extension assays were used to assess the incorporation of multi 2′F-araATP or 2′F-araGTP units (8×2′F-araA and 11× 2′F-araG) on the DNA-FANA chimeric template PF34. Two-three folds of cold DNA primer (PF20) was combined with the ³²P-labeled DNA primer (PF20), and mixed together with the DNA template strand PF34 in a 1:3 molar ratio (final concentration: 85 nM primer: 255 nM template). The resulting mixture was heated at 95° C. for 5 min, and annealed at 4° C. for at least two hours before use. In an Eppendorf tube, equimolar concentrations nucleoside 5′-triphosphates (2′F-araATP or 2′F-araGTP plus other three dNTPs at final concentration 0.4 mM) were combined with the primer and template mixture according to the conditions listed on Table 2 and FIG. 5. Mineral oil (20 μL) was used to prevent evaporation. The reaction mixture was incubated at either 37° C. (for KF) or 55° C. (for other polymerases) and different enzyme was added to initiate the primer extension reaction. Time points were obtained by small aliquots (4 μL or 8 μL) from the reaction mixture and quenching the aliquot with the same volume of a stopping dye solution (98% deionized formamide, 10 mM EDTA, 1 mg/ml bromophenol blue and 1 mg/ml xylene cyanol). The product pattern from each time point was analyzed by 12% denaturing polyacryamide gel electrophoresis (PAGE) and subsequent autoradiography.

Under these conditions, all the enzymes studied could incorporate four 2′F-araATP (FIG. 9A) and 6 2′F-araGTP (FIG. 9B) before the enzyme reached a 2′F-araN unit on the template (within the running start sequence in the template PF34). Consistent with previous examples, the thermophilic enzymes DV and 9N were even able to incorporate the 2′F-araATPs or 2′F-araGTPs on a FANA-DNA chimeric template, much more efficiently than other polymerases (Bst, Taq, KF). Another thermophilic enzyme, Therminator (Th), a mutated 9N DNA polymerase, could also give full-length product as effectively as DV and 9N. Taq did not afford full-length products and KF and Bst could generate full-length products but with strong pausing phenomenon (chain termination). Strong pausing was observed with Kf and Bst after the running start sequence. Pausing was observed earlier for Taq after the first incorporation of 2′F-araGTP.

This example clearly shows that multi 2′F-araA and 2′F-araG units can be incorporated by certain DNA polymerases (e.g. DV, 9N and Th) not only on a DNA template (i.e. segment 3′- . . . CCCTCTTCTC . . . -5′ of template PF34) but on a chimeric DNA-FANA segment as well.

### Example 10

### Polymerase-Directed Synthesis of 2′-Deoxy-2′-Fluoro-β-D-Arabino-Nucleic Acids (2′F-ANA) of Mixed Base Composition

Primer extension assays were used to screen various DNA polymerases for their ability to incorporate 2′F-araATP, 2′F-araCTP, 2′F-araGTP, and 2′F-araTTP to make a mixed FANA sequence. Family B polymerases (DV, 9N, Th, Ph) were shown to effectively incorporate all four 2′F-araNTPs to yield full-length FANA products (FIG. 10), whereas family A polymerases (Bst, Taq, Kf) and MMLV generated premature products containing only two to four 2′F-araNTP residues.

In summary, family B thermophilic DNA polymerases such as DV, 9N and Ph can utilize 2′F-araNTPs, or a combination of 2′F-araNTPs and dNTPs, to generate FANA or chimeric FANA-DNA strands, respectively. In addition, these polymerases were shown to synthesize chimeric FANA-DNA strands on a chimeric FANA-DNA template. KF and Bst (family A) DNA polymerases were able to incorporate dNTPs on a template FANA strand.

### Example 11

### Fidelity of DNA Polymerases in the Presence of 2′F-araNTPs

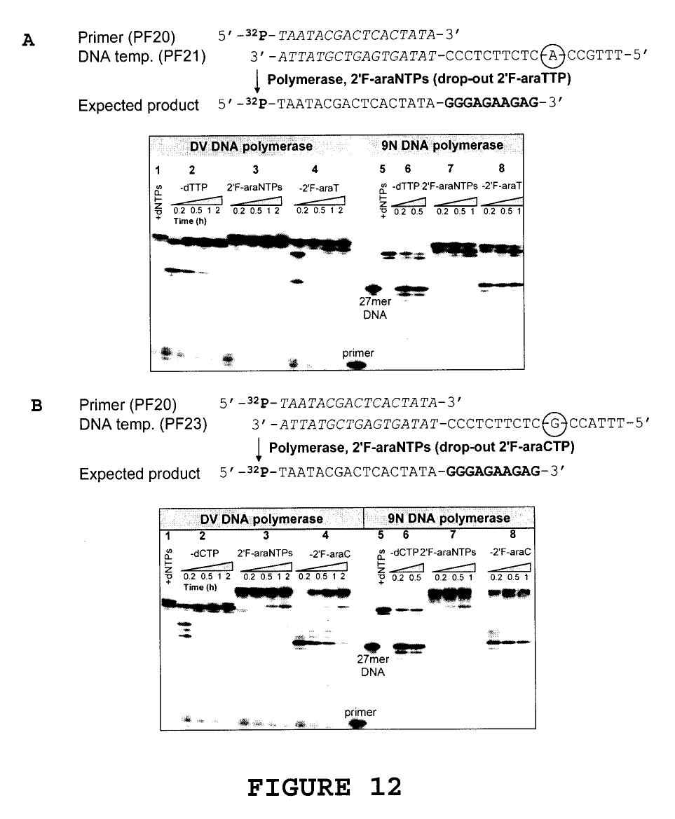

“Dropout assays” (Ichida et al. (2005) J. Am. Chem. Soc. 127:2802-2803) were conducted to assess apparent fidelity of 2′F-araNTP incorporation by DV and 9N. In one of these assays, 2′F-araATP was removed from the pool, and the ensuing synthesis assessed in comparison with a control reaction containing all four 2′F-araNTPs. For comparison, dropout experiments containing dNTPs were run in parallel (FIGS. 11-14). Full-length DNA and FANA products were obtained when all four dNTPs (group 1 and 5) and 2′F-araNTPs (groups 3 and 7) were available, although some pausing was evident during synthesis of the arabinose modified oligomers. Compared with DATP, both DV and 9N (FIGS. 11 and 12) exhibited excellent selectivity on 2′F-araATP, as demonstrated by efficient chain termination at the position where 2′F-araATP was required (groups 4 and 8). In contrast, dropping out DATP in these assays (groups 2 and 6) afforded full-length in addition to premature products (Table 4), suggesting that 2′F-araATP is more strictly selected than DATP under these conditions.

TABLE 4
Apparent fidelity of FANA synthesis in dropout assay

| | % Apparent Fidelity^{a} | | Template | |
|---|---|---|---|---|
| Polymerase | 2′F-araNTP | dNTP | used | FIG. |
| Ph | A: >99% | >99% | PF21 | 13 |
| | G: >99% | >99% | PF41 | 14 |
| | T: >99% | >99% | | |
| | C: >99% | >99% | | |
| DV | A: >99% | 1% | PF41 | 11A |
| | G: >99% | 62% | PF43 | 11B |
| | T: 3% | 19% | PF21 | 12A |
| | C: 50% | 3% | PF23 | 12B |
| 9N | A: >99% | 80% | PF41 | 11A |
| | G: 98% | 84% | PF43 | 11B |
| | T: 76% | 32% | PF21 | 12A |
| | C: 43% | 82% | PF23 | 12B |

^{a}Apparent fidelity is calculated according to the equation 1 − [(% full-length − 2′F-araNTP)/(% full-length + 2′F-araNTP)] at 30 min reaction time (sample equation for dNTP values); >99% means that the full-length product is not detectable in our dropout assays.

The same dropout experiments were used to evaluate fidelity of the remaining three triphosphates, namely, 2′F-araGTP, 2′F-araCTP and 2′F-araTTP (Table 4; FIGS. 11B-12). DV and 9N demonstrated higher fidelity with respect to 2′F-araGTP incorporation relative to the native dGTP (FIG. 11B). Selectivity toward the pyrimidine-based 2′F-araNTPs was significantly less, but the same was true for the corresponding pyrimidine-containing dNTPs. The selectivity for 2′F-araNTP incorporation by DV and 9N polymerases appeared to be comparable, if not better, than that for dNTPs.

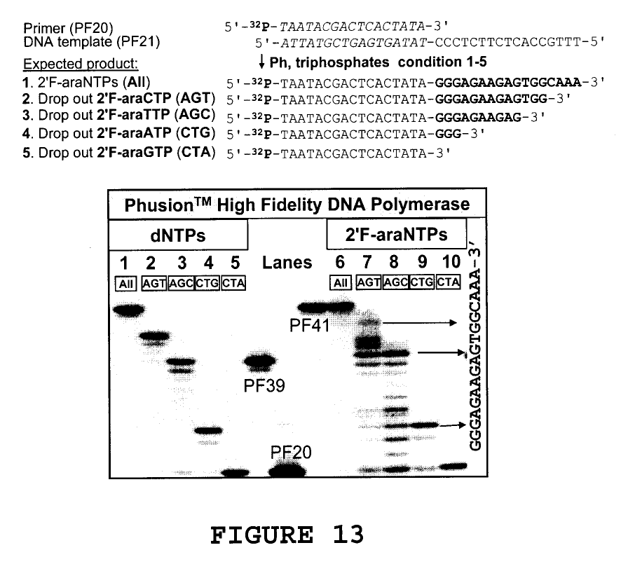

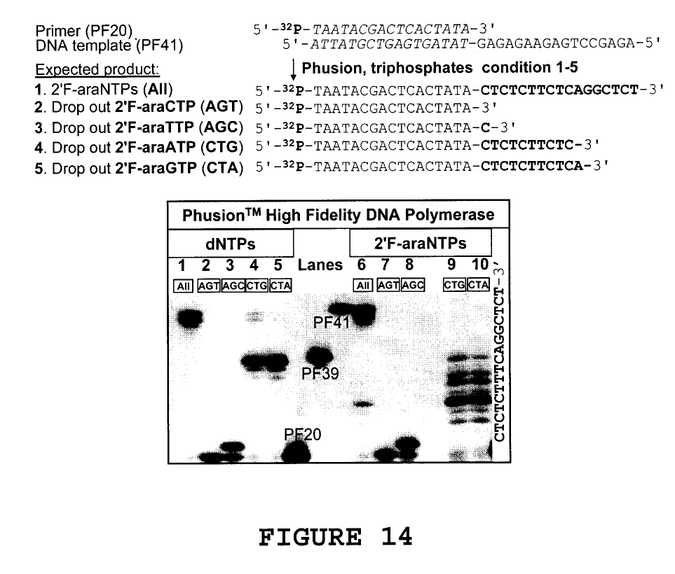

Phusion DNA polymerase (Ph) was evaluated for fidelity of 2′F-araNTP incorporation using the same dropout assay. All four dropout experiments were conducted with the same DNA template PF21 (FIG. 13), or PF41 (FIG. 14). For the dNTPs, syntheses stopped at the expected termination sites, yielding products lacking the required dNTP at the 3′-terminus. A similar pattern was observed with 2′F-araNTPs (FIG. 13; lanes 6-10). This effect, however, was observed only in the dropout assays, that is, the reaction containing all four 2′F-araNTPs produced excellent yields of the full-length FANA product with virtually no pausing observed. Similar results were observed with the DNA template PF41 (FIG. 14).

### Example 12

### 2′F-arabino Versus 2′F-ribonucleoside 5′-triphosphates as Substrates of DNA Polymerases

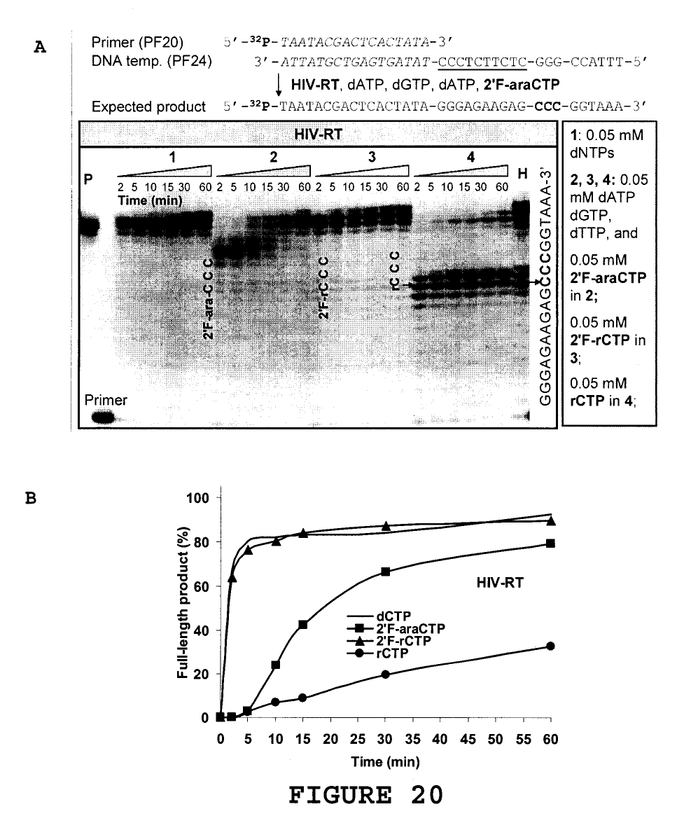

The primer extension assays were used to assess the incorporation efficiency of three contiguous 2′F-ara(T/C)TP versus 2′F-r(U/C)TP on a DNA template (PF22 or PF24). To this end, template PF22 (FIGS. 15, 16, 17A, 18 and 21A) or template PF24 (FIGS. 17B, 19, 20 and 21B); primer PF20; and the appropriate NTPs: dATP/dGTP/dCTP/ and NTP, where N=dT, 2′F-araT, 2′F-rU, or rU; or dATP/dGTP/dTTP/ and NTP, where N=dC, 2′F-araC, 2′F-rC, or rC; were incubated in the presence HIV (FIGS. 18 and 20), DV (FIG. 21), MMLV-RT (FIGS. 15, 16, and 19) or 9N (FIG. 17) following the protocols and conditions described in Example 2 and Table 2.

The results show that 2′F-araTTP is an excellent substrate of HIV under the conditions used (FIG. 18A), and its incorporation proceeded more efficiently compared to 2′F-rUTP and rUTP (FIG. 18B). Different pausing patterns were observed for the various NTPs. Delayed pausing is observed after the last 2′F-araTTPs was incorporated, whereas for the rNTPs (i.e., 2′F-rUTP or rUTP), strong pausing occurred prior or immediately after incorporation of the first rNTP (FIG. 18A).

Similar results were obtained using MMLV-RT, which was shown to incorporate 2′F-araTTP more efficiently than 2′F-rUTP and rUTP (FIGS. 15 and 16). Pausing was observed after the first incorporation of 2′F-araTTP, and before the first incorporation of 2′F-rUTP and rUTP. Analysis of the second incorporations of modified triphosphates (FIG. 16) demonstrated that 2′F-araTTP was the best substrate for MMLV-RT.

The efficiency of incorporation of 2′F-araTTP and 2′F-rUTP by the thermostable DV polymerase was determined (FIG. 21A). Similar to results obtained with 9N polymerase (FIG. 17A), incorporation of 2′F-araTTPs proceeded efficiently and provided a near quantitative yield of full-length product after 5 minutes, with accumulation of an intermediate band corresponding to 5′-[DNA primer]-2′F-ara(TpTpT)-dG-3′ within the first 2 minutes (FIG. 21A). With 2′F-rUTP, however, abrupt pausing was observed after the first and second 2′F-rUTP incorporation, yielding only 20% of the desired full-length product after 9.5 h. Incorporation efficiencies of 2′F-araTTP and dTTP were comparable and ca. 100% full length products were generated within 2 mins. without pausing, compared to reactions with 2′F-rUTP which showed pausing after one incorporation producing little of the expected full-length product (ca. 20% after one hour).

The same primer extension assays were used to evaluate the efficiency of DNA-template (PF24) directed incorporation of the cytosine NTPs by 9N polymerase (FIG. 17B); MMLV-RT (FIG. 19), HIV (FIG. 20) and DV polymerase (FIG. 21B). The incorporation of 2′F modified CTPs (FIGS. 19-21) seemed to be more complicated than what was previously shown with 2′F modified TTPs. Generally, in contrast to what was observed for the thymine NTPs, the incorporation efficiency of 2′F-araCTP by HIV (FIG. 20B) and MMLV (FIG. 19B) is comparable to or slightly less efficient than that observed with 2′F-rCTP but better than rCTP. 2′F-rCTP behaved very differently from rCTP, showing little or no pausing with either of these polymerases, whereas rCTP showed significant pausing before the first rCTP incorporation, as observed with rUTP.

In the primer extension assay using DNA template PF24 (9N: FIG. 17B; HIV: FIG. 20B; MMLV: FIG. 19; DV: FIG. 21B), incorporation efficiency followed the order: dCTP=2′F-rCTP>2′F-araCTP >>rCTP. Nevertheless, significant amount of full length products are observed for both 2′F-araCTP and 2′F-rCTP incorporations.

Binding of 2′F-araCTP at the active site of MMLV-RT appeared to occur more readily than binding of 2′F-rCTP, its isomeric counterpart, as evidenced by pausing after the first 2′F-araCTP incorporation but before the first 2′F-rCTP incorporation (FIG. 19). Further elongation beyond the 2′F-araC unit, however, was difficult and mainly premature products (ca. 90%) were observed after 60 mins. In contrast, once 2′F-rCTP was incorporated, synthesis proceeded readily giving rise to almost 100% of full-length product (FIG. 19). Both DV (FIG. 21B) and 9N (FIG. 17B) were able to incorporate 2′F-araCTP as well as 2′F-rCTP under the conditions studied.

In summary, incorporation of 2′F-araTTP proceeded more efficiently relative to 2′F-rUTP, while the incorporation of 2′F-araCTP was slightly less efficient than that observed with 2′F-rCTP. The substrate specificity towards the 2′F-rUTP and rUTP pair were similar but had different specificity towards the corresponding cytosine pair (i.e. 2′F-rCTP and rCTP). The order of incorporation of efficiency of 2′F modified pyrimidine NTPs is summarized in Table 5.

TABLE 5
The order of incorporation efficiency of 2′F-araT(C)TP vs 2′F-rU(C)TP

| Polymerase | Order of efficiency of incorporation | Figures |
|---|---|---|
| HIV | dTTP > 2′F-araTTP > 2′F-rUTP > UTP | 18 |
| | dCTP~2′F-rCTP ≧ 2′F-araCTP > CTP | 20 |
| MMLV-RT | dTTP > 2′F-araTTP > 2′F-rUTP > UTP | 15, 16 |
| | dCTP~2′F-rCTP ≧ 2′F-araCTP > CTP | 19 |
| DV | dTTP~2′F-araTTP > 2′F-rUTP | 21A |
| | dCTP~2′F-rCTP~2′F-araCTP | 21B |
| 9N | dTTP~2′F-araTTP > 2′F-rUTP | 17A |
| | dCTP~2′F-araCTP~2′F-rCTP | 17B |

### Example 13

### Enzymatic Synthesis of Chimeric Oligonucleotides Containing Both 2′F-arabino and 2′F-ribononucleosides

The method described here provides the first examples of chimeric oligonucleotides containing both 2′F-ara and 2′F-ribonucleosides. Polymerization is carried out using the ³²P-primer (PF20)/template (PF32) pair, and various NTP combinations following the protocol described in Example 2 and Table 2. More specifically, the primer/template pair PF20/PF32 was incubated in the presence of 9N, DV or HIV DNA polymerase, DATP, dGTP and one of four pyrimidine pairs: (a) 2′F-araTTP/2′F-araCTP; (b) 2′F-araTTP/2′F-rCTP; (c) 2′F-rUTP/2′F-rCTP; and (d) rUTP/rCTP. The results are shown in FIGS. 22-24 and demonstrate that 9N polymerase can incorporate 2′F-araTTP/2′F-araCTP and 2′F-araTTP/2′F-rCTP much more efficiently than 2′F-rUTP/2′F-rCTP, 2′F-rUTP/2′F-araCTP, and rUTP/rCTP under these conditions (FIG. 22).

Incorporation efficiency was measured either as the percentage of full-length products, or the number of nucleotides incorporated if no full-length products were obtained. With 9N (FIG. 22) and DV polymerases (FIG. 23), incorporation efficiencies followed the same trend (Table 5). HIV (FIG. 24) produced the opposite results for Groups 3, 4 and 6 (Table 5). Generally, dNTPs were the best substrate and rNTPs the worst substrates while combinations of 2′F modified NTPs showed different incorporation efficiencies (Table 5).

TABLE 5
The order of efficiency of incorporation of different triphosphate pairs.

| Polymerase | Order of efficiency of incorporation | Figure |
|---|---|---|
| 9N | All four dNTPs (group 1) > | 22 |
| | 2′F-araTTP/2′F-araCTP (group 3) > | |
| | 2′F-araTTP/2′F-rCTP (group 4) > | |
| | 2′F-rUTP/2′F-rCTP (group 6) > | |
| | 2′F-rUTP/2′F-araCTP (group 5)~ | |
| | rUTP/rCTP (group 7) >> | |
| | All four rNTPs (group 2) | |
| DV | All four dNTPs (group 1)~ | 23 |
| | 2′F-araTTP/2′F-araCTP (group 3)~ | |
| | 2′F-araTTP/2′F-rCTP (group 4) > | |
| | 2′F-rUTP/2′F-rCTP (group 6) > | |
| | 2′F-rUTP/2′F-araCTP (group 5) > | |
| | rUTP/rCTP (group 7) > | |
| | All four rNTPs (group 2) | |
| HIV | All four dNTPs (group 1) >> | 24 |
| | 2′F-rUTP/2′F-rCTP (group 6) > | |
| | 2′F-araTTP/2′F-rCTP (group 4) > | |
| | 2′F-araTTP/2′F-araCTP (group 3)~ | |
| | 2′F-rUTP/2′F-araCTP (group 5)~ | |
| | rUTP/rCTP (group 7) > | |
| | All four rNTPs (group 2) | |

A summary of the results of Examples 1 to 13 are shown in the following Table 6.

Although preferred embodiments of the invention have been described herein, it will be understood by those skilled in the art that variations may be made thereto without departing from the spirit of the invention or the scope of the appended claims. All documents referenced herein are hereby incorporated by reference.

TABLE 6
Performance of DNA polymerases tested in primer extension reactions^{a}

| Example # & | | | | | | | | | | | |
|---|---|---|---|---|---|---|---|---|---|---|---|
| Template # | Type and function | Triphosphates | DV | 9N | Bst | Taq | Ph | Kf | MMLV | HIV | Th |
| 2 | DNA template, | 2′F-araTTP, | +++^{b} | +++^{b} | +++ | ++^{b} | ++++ | ++ | +++ | ++++ | n.d. |
| PF22 | 3x 2′F-araTTP | dATP, dGTP, dCTP | | | | | | | | | |
| 3 | DNA template, | 2′F-araCTP, | ++++ | +++ | ++++ | ++++ | ++++ | ++ | +++ | ++++ | n.d. |
| PF24 | 3x 2′F-araCTP | dATP, dGTP, dTTP | | | | | | | | | |
| 4 | DNA template, | 2′F-araTTP, 2′F- | ++++ | ++++ | ++++ | ++++ | ++++ | +++ | n.d. | +++ | n.d. |
| PF32 | 3x 2′F-araT + | araCTP | | | | | | | | | |
| | 3x 2′F-araC | dATP, dGTP | | | | | | | | | |
| 5 | DNA template, | 2′F-araTTP, 2′F- | ++++ | ++++ | ++ | + | ++ | + | n.d. | + | n.d. |
| PF33 | 6x 2′F-araT + | araCTP, dATP, | | | | | | | | | |
| | 6x 2′F-araC | dGTP | | | | | | | | | |
| 6 | FANA-DNA | dNTPs | ++++ | ++++ | ++++ | ++++ | ++ | ++++ | n.d. | ++++ | n.d. |
| PF34 | template(40%) | 2′F-araTTP, 2′F- | +++ | +++ | + | + | + | + | n.d. | + | n.d. |
| | | araCTP, dATP, | | | | | | | | | |
| | | dGTP | | | | | | | | | |
| 7 | FANA-DNA | dNTPs | +++ | ++++ | ++++ | ++++ | + | ++++ | n.d. | +++ | n.d. |
| PF35 | template(60%) | 2′F-araTTP, 2′F- | + | + | + | + | + | + | n.d. | + | n.d. |
| | | araCTP, dATP, | | | | | | | | | |
| | | dGTP | | | | | | | | | |
| 8 | FANA-DNA | dNTPs | + | + | ++ | + | + | ++ | n.d. | + | n.d. |
| PF31 | template(100%) | | | | | | | | | | |
| 9 | FANA-DNA template | 2′F-araATP or | ++++ | ++++ | + | + | ++++ | +++ | n.d. | n.d. | ++++ |
| PF34 | (40%), 8x 2′F-araA | 2′F-araGTP plus | | | | | | | | | |
| | or 11x 2′F-araG | other three dNTPs | | | | | | | | | |
| 10 | DNA template | 2′F-araNTPs | ++++ | ++++ | + | + | ++++ | + | + | n.d. | ++++ |
| PF33 | Mixed FANA | | | | | | | | | | |
| | sequences | | | | | | | | | | |
| | containing 7X 2′F- | | | | | | | | | | |
| | araG, 11X 2′F- | | | | | | | | | | |
| | araA, 6X 2′F- | | | | | | | | | | |
| | araT, 6X 2′F-araC | | | | | | | | | | |

Note:
^{a}Enzymatic activity during primer extension reactions: ++++ excellent (no pausing observed) +++ very good (with slight pausing); ++ Good, full-length products with significant pausing; + significant pausing, no full-length product but at least one 2′F-araN incorporation detected;
^{b}at 37° C. (see Table 2 for temperatures of all other reactions);
n.d.: not determined

1. A method for performing polymerase-directed oligonucleotide synthesis comprising:
a) providing a template oligonucleotide;
b) providing a primer for the template oligonucleotide;
c) providing monomers of nucleoside-5′-triphosphates; and
d) a polymerase,
wherein at least one nucleoside of at least one of (i) the template oligonucleotide and (ii) the monomers is a 2′-deoxy-2′-fluoroarabinonucleoside (2′F-araN).

2. The method of claim 1, wherein the template oligonucleotide is DNA.

3. The method of claim 2, wherein at least one monomer is a 2′-deoxy-2′-fluoroarabinonucleoside 5′-triphosphate (2′F-araNTP).

4. The method of claim 3, wherein all monomers having a predetermined base are 2′F-araNTPs.

5. The method of claim 3, wherein the monomers are a mixture of 2′-deoxynucleoside 5′-triphosphates (dNTPs) and 2′F-araNTPs.

6. The method of claim 3, wherein all monomers are 2′F-araNTPs.

7. The method of claim 1, wherein the template oligonucleotide comprises at least one 2′F-araNTP.

8. The method of claim 7, wherein the template oligonucleotide consists of 2′-deoxy-2′-fluoroarabinonucleotides (FANA).

9. The method of claim 7, wherein the template oligonucleotide is a FANA-DNA chimera with a FANA percentage of less than 100%.

10. The method of claim 9, wherein the FANA percentage is less than 60%.

11. The method of claim 10, wherein the FANA percentage is less than 40%.

12. The method of claim 7, wherein the monomers are dNTPs.

13. The method of claim 7, wherein at least one monomer is a 2′F-araNTP.

14. The method of claim 13, wherein all monomers having a predetermined base are 2′F-araNTPs.

15. The method of claim 13, wherein the monomers are a mixture of dNTPs and 2′F-araNTPs.

16. The method of claim 13, wherein the monomers are a mixture of rNTPs and 2′F-araNTPs.

17. The method of claim 13, wherein all monomers are 2′F-araNTPs.

18. The method of claim 1, wherein the polymerase is selected from the group consisting of DV, 9N, Bst, Th, Taq, Ph, Kf, MMLV and HIV.

19. The method of any one of claim 1 wherein the monomers comprises at least one modified nucleoside 5′-triphosphate.

20. The method of claim 19, wherein the at least one modified nucleoside triphosphate is selected from the group consisting of ribonucleoside 5′-(alpha-P-borano)-triphosphates (BH₃-RNA), ribonucleoside 5′-(alpha-thio)triphosphates (S-RNA), 2′-deoxyribonucleoside 5′-(alpha-methyl)triphosphates (P-Me DNA), alpha-L-threofuranosyl nucleoside 5′-triphosphates (TNA), 4′-thio-ribonucleoside 5′-triphosphates (4′S-RNA), 2′-amino-ribonucleoside-5′triphosphate (2′NH₂-RNA), 2′-deoxy-2′-fluororibonucleoside-5′-triphosphate (2′F-RNA) and combinations thereof.

21. The method of claim 1, wherein the 2′F-araN unit comprises any heterocyclic base capable of Watson-Crick base pairing.

22. The method of claim 21, wherein the base is selected from the group consisting of thymine, uracil, cytosine, adenine, guanine, inosine, 2,6-diaminopurine, 5-methylcytosine, 5-fluorocytosine, 5-bromocytosine, 5-iodocytosine, isocytosine, N⁴-methylcytosine, 5-iodouracil, 5-fluorouracil, 4-thiouracil, 4-thiothymine, 2-thiouracil, 2-thiothymine, 7-deaza-adenine, N⁶-methyladenine, isoguanine, 7-deaza-guanine, 6-thioguanine, and combinations thereof.

23. A method for performing SELEX comprising:
a) providing a library of oligonucleotides;
b) selecting the library for binding to a target molecule to produce a binding population; and
c) amplifying the binding population, said amplifying step comprising a synthesis step comprising the method of claim 1.

24. A method of generating a library of oligonucleotides comprising a synthesis step comprising the method of claim 1.

25. A method for performing SELEX comprising:
a) providing a library generated by the method of claim 24;
b) selecting the library for binding to a target molecule to produce a binding population; and
c) amplifying the binding population.

26-28. (canceled)
